(19) United States
(12) Patent Application Publication
(10) Pub. No.: US 2005/0113370 A1
(43) Pub. Date: May 26, 2005

(54) Therapeutic substituted indazole derivatives

(76) Inventors: Jonas Malmstrom, Sodertalje (SE); Britt-Marie Swahn, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/504,411

(22) PCT Filed: Feb. 11, 2003
(86) PCT No.: PCT/SE03/00227

(30) Foreign Application Priority Data
Feb. 13, 2002 (SE) 0200450-5
Oct. 22, 2002 (SE) 0203122-7

(51) Int. Cl. A61K 31/541; A61K 31/5377; A61K 31/496; A61K 31/454; A61K 31/416; C07D 417/02; C07D 413/02

(52) U.S. Cl. 514/227.8; 514/232.5; 514/254.06; 514/320; 514/406; 544/60; 544/140; 544/371; 546/199; 548/361.1

(57) ABSTRACT

The invention provides a compound of Formula I Formula I wherein R¹ is aryl or heteroaryl each of which is optionally substituted with one or more of the following R³, —OR³, —OCOR³, —COOR³, —COR³, —CONR³R⁴, —NHCOR³, —NR³R⁴, —NHSO₂R³, —S₂R³, —SO₂NR³R⁴, —SR³, CN, halogeno or NO₂; R² is NO₂, NH₂, —NR⁵R⁶ or —NR⁶R⁷; as a free base or a pharmaceutically acceptable salt, solvate or solvate of salt thereof, a process for their preparation, pharmaceutical formulations containing said compounds and to the use of said compounds in therapy.

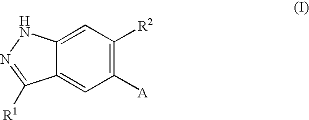

### TECHNICAL FIELD

The present invention relates to novel substituted indazole derivatives, useful for treatment of various disorders. The invention relates to methods for producing these compounds. The invention also provides pharmaceutical compositions comprising the compounds of the invention and methods of utilizing these compositions in the treatment of various disorders.

### BACKGROUND TO THE INVENTION

Protein kinases are important components of intracellular signalling pathways and kinases are involved in the regulation of a variety of cellular functions. The MAP kinase signalling pathways are activated by engagement of a number of cell surface receptors. One of these pathways, the JNK pathway is activated specifically by stress or pro-inflammatory cytokines. Activators include LPS, the cytokines tumour necrosis factor (TNF-α) and Interleukin-1 (IL-1), osmotic shock, chemical stress and UV radiation (Cohen, P. Trends in Cell Biol. 7:353-361 1997). Targets of the JNK pathway include a number of transcription factors, such as but not exclusively c-jun and ATF-2 (Whitmarsh, A. and Davis, R. J. Mol. Med. 74:589-607 1998).

Three different genes: JNK1, JNK2 and JNK3; encode the JNK family of enzymes. Alternatively spliced forms of these genes can give rise to 10 distinct isoforms: four for JNK1, four for JNK2 and two for JNK3. (Gupta, S. et al EMBO J. 15:2760-2770 1996). JNK1 and JNK2 are ubiquitously expressed in human tissues whereas JNK3 is selectively expressed in the brain, heart and testis (Dong, C. et al. Science 270:1-4 1998).

JNKs 1, 2 and 3 have been selectively knocked out in mice both singulary and in combination by both gene deletion and/or transgenic expression of dominant negative forms of the kinases (Dong, C. et al Science 282:2092-2095 1998; Yang, D. et al Immunity 9:575-585 1998; Dong, C., et al Nature 405:91-94 2000; Yang, D. et al Nature 389:865-870 1997). Mice with targeted disruption of the JNK3 gene develop normally and are protected from excitotoxin induced apoptosis of neurones. This finding suggests that specific inhibitors of JNK3 could be effective in the treatment of neurological disorders characterised by cell death such as Alzheimer's disease and stroke. Mice disrupted in either JNK1 or 2 also develop normally. Peripheral T cells from either type of mice can be activated to make IL2, but in both cases, there is a defect in Th1 cell development. In the case of JNK1 −/− mice, this is due to an inability to make gamma interferon (a key cytokine essential for the differentiation of Th1 cells). In contrast, JNK2 −/− mice produce interferon gamma but are unable to respond to the cytokine. Similar defects in T cell biology (normal IL2 production but a block in Th1 cell differentiation) are seen in T cells disrupted in the MKK7 gene confirming this role for the JNK pathway in T cell differentiation (Dong, C., et al Nature 405:91-94 2000).

JNK also plays a major role in apoptosis of cells (Davis R J. Cell. 103:239-252 2000). JNK is essential for UV induced apoptosis through the cytochrome C mediated pathway (Tournier, C. et al Science 288:870-874 2000). Ischemia and ischemia coupled with re-perfusion as well as restricted blood flow itself have been shown to be accompanied by activation of JNK. Cell death can be prevented with dominant negative forms of JNK transfected into cells demonstrating a potential utility for JNK in conditions characterised by stress-induced apoptosis.

Activation of the JNK pathway has been observed in a number of human tumours and transformed cell lines (Davis R J. Cell. 103:239-252 2000). Indeed, one of the major targets of JNK, c-jun, was originally identified as an oncogene indicating the potential of this pathway to participate in unregulated cell growth. JNK also regulates phosphorylation of p53 and thus modulates cell cycle progression (Chen T. et al Mol. Carcinogenesis 15:215-226 1996). Inhibition of JNK may therefore be beneficial in some human cancers.

Based on current knowledge of JNK signalling, especially JNK3, has been implicated in areas of neurodegenerative diseases such as Alzheimer's disease, Parkinson's disease, ALS, Huntington's disease, traumatic brain injury, as well as ischeric and haemorrhaging stroke.

Thus, there is a high unmet medical need for JNK specific inhibitors useful in treating the various conditions associated with JNK activation.

Superficially similar compounds are known in the art, for example in WO 02/10137, WO 00/44728, WO 97/03069, and in Kawami et al. (Org. Lett. Vol. 2, No. 3, 2000, p 413-415).

### DISCLOSURE OF THE INVENTION

It has been found that compounds of the Formula I, which are aryl substituted heterocyclic compounds, are particularly effective JNK inhibitors and thereby suitable in the treatment of the various conditions associated with JNK activation.

In one aspect, the invention thus relates to compounds of Formula I 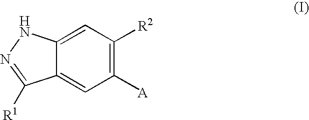
wherein:
R¹ is aryl or heteroaryl each of which is optionally substituted with one or more of the following R³, —OR³, —OCOR³, —COOR³, —COR³, —CONR³R⁴, —NHCOR³, —NR³R⁴,
—NHSO₂R³, —SO₂R³, —SO₂NR³R⁴, —SR³, CN, halogeno or NO₂;
R² is NO₂, NH₂, —NR⁵R⁶ or —NR⁶R⁷;
R³ and R⁴ are each independently hydrogen, C₁₋₆alkyl, C₂₋₆alkenyl, (C₃₋₈cycloalkyl)C₀₋₆alkyl, C₁₋₆fluoroalkyl, heterocycleC₀₋₆alkyl, heteroarylC₀₋₆alkyl; and said C₁₋₆alkyl, C₂₋₆alkenyl, (C₃₋₈cycloalkyl)C₀₋₆alkyl, C₁₋₆fluoroalkyl, heterocycleC₀₋₆alkyl, heteroarylC₀₋₆alkyl may be substituted with one or more B;
or R³ and R⁴ form together a 5-, 6- or 7-membered heterocyclic ring containing 1 to 4 heteroatoms independently selected from N, O and S, and said ring may be substituted with one or more B;
B is R¹⁰, —COOR¹⁰, —COR¹⁰, —NHCOR¹⁰, —NR¹⁰R¹¹, —CONR¹⁰R¹¹, —OR¹⁰, —SO₂NR¹⁰R¹¹, CN, halogeno or oxo;
R⁵ is phenyl or heteroaryl each of which is optionally substituted with one or more of R¹⁰, —OR¹⁰, —OCOR¹⁰, —COOR¹⁰, —CONR¹⁰R¹¹, —NHCOR¹⁰, —NR¹⁰R¹¹, —NHSO₂R¹⁰, —SO₂R¹⁰, —SO₂NR¹⁰R¹¹, —SR¹⁰, CN, halogeno, or NO₂;
R⁶ is hydrogen, C₁₋₆alkyl, heterocycleC₀₋₆alkyl, or hydroxyC₁₋₆alkyl;
R⁷ is C₁₋₆alkyl, (C₃₋₈cycloalkyl)C₀₋₆alkyl, C₅₋₈cycloalkenylC₀₋₆alkyl, or R⁵C₁₋₆alkyl;
A is hydrogen, R⁸, —OR⁸, —OCOR⁸, —COOR⁸, —CONR⁸R⁹, —NHCOR⁸, —NR⁸R⁹, —NHSO₂R⁸, —SO₂R⁸, —SO₂NR⁸R⁹, —SR⁸, CN, halogeno, heterocycleC₀₋₆alkyl, or heteroarylC₀₋₆alkyl;
R⁸ and R⁹ each independently are hydrogen, C₁₋₆alkyl, C₂₋₆alkenyl, C₂₋₆alkynyl heterocycleC₀₋₆alkyl-, heteroarylC₀₋₆alkyl; and said C₁₋₆alkyl, C₂₋₆alkenyl, C₂₋₆alkynyl heterocycleC₀₋₆alkyl, or heteroarylC₀₋₆alkyl may be substituted with one or more B;
or R⁸ and R⁹ form together a 5-, 6- or 7-membered heterocyclic ring containing 1 to 4 heteroatoms independently selected from N, O and S, and said ring may be substituted with one or more B;
R¹⁰ and R¹¹ each independently are hydrogen, C₁₋₆alkyl, C₁₋₆fluoroalkyl or hydroxyC₁₋₆alkyl, or;
R¹⁰ and R¹¹ form together a 5-, 6- or 7-membered heterocyclic ring containing 1 to 4 heteroatoms independently selected from N, O and S, and said ring may be substituted with one or more B;
with the proviso that said compound is not 6-amino-3-(4-fluorophenyl)-indazole, 6-amino-3-phenyl-indazole, 6-nitro-3-phenyl-indazole, 6-nitro-3-(4-nitrophenyl)-indazole and that said compounds has not a quinazoline in R⁵ position;
   as a free base or a salt thereof.

Prefarably, the compounds of formula I are present in the form as a pharmaceutically acceptable salt.

Listed below are definitions of various terms used in the specification and claims to describe the present invention.

For the avoidance of doubt it is to be understood that where in this specification a group is qualified by ‘hereinbefore defined’ or ‘defined hereinbefore’ the said group encompasses the first occurring and broadest definition as well as each and all of the preferred definitions for that group.

For the avoidance of doubt it is to be understood that in this specification ‘C₀₋₆’ means a carbon group having 0, 1, 2, 3, 4, 5 or 6 carbon atoms.

In this specification, unless stated otherwise, the term “alkyl” includes both straight and branched chain alkyl groups. C₁₋₆alkyl may be methyl, ethyl, n-propyl, i-propyl, n-butyl, i-butyl, s-butyl, t-butyl, n-pentyl, i-pentyl, t-pentyl, neo-pentyl, and hexyl.

In this specification, unless stated otherwise, the term “C₃₋₈ cycloalkyl” includes a non-aromatic, completely saturated cyclic aliphatic hydrocarbon group containing 3 to 8 atoms. Examples of said cycloalkyl include, but are not limited to cyclopropyl, cyclobutyl, cyclopentyl and cyclohexyl.

The term “alkoxyl” as used herein, unless stated otherwise includes “alkyl” O groups in which “alkyl” is as hereinbefore defined. C₁₋₆alkoxyl may be methoxy, ethoxy, n-propoxy, i-propoxy, n-butoxy, i-butoxy, s-butoxy, t-butoxy, n-pentyloxy, i-pentyloxy, t-pentyloxy, neo-pentyloxy, hexyloxy.

In this specification, unless stated otherwise, the term “alkenyl” includes both straight and branched chain alkenyl groups but references to individual alkenyl groups such as 2-butenyl is specific for the straight chain version only. Unless otherwise stated, the term “alkenyl” advantageously refers to chains with 2 to 5 carbon atoms, preferably 3 to 4 carbon atoms. C₂₋₆alkenyl may be, but is not limited to, ethenyl, propenyl, 2-methylpropenyl, butenyl and 2-butenyl.

In this specification, unless stated otherwise, the term “alkynyl” includes both straight and branched chain alkynyl groups but references to individual alkynyl groups such as 2-butynyl is specific for the straight chain version only. Unless otherwise stated, the term “alkynyl” advantageously refers to chains with 2 to 5 carbon atoms, preferably 3 to 4 carbon atoms.

In this specification, unless stated otherwise, the term “heterocycle” includes a 3- to 10-membered non-aromatic partially or completely saturated hydrocarbon group, which contains one or two rings and at least one heteroatom. Examples of said heterocycle include, but are not limited to pyrrolidinyl, pyrrolidonyl, piperidinyl, piperazinyl, morpholinyl, oxazolyl, 2-oxazolidonyl or tetrahydrofuranyl.

In this specification, unless stated otherwise, the expression “NR³R⁴ can form a ring having 5, 6 or 7 atoms, said ring optionally including one or more additional heteroatoms selected from N, O or S” include, but are not limited to piperidinyl, piperazinyl and morpholinyl.

In this specification, unless stated otherwise, the term “aryl” may be a C₆-C₁₄ aromatic hydrocarbon and includes, but is not limited to, benzene, naphthalene, indene, anthracene, phenanthrene.

In this specification, unless stated otherwise, the term “heteroaryl” may be a monocyclic heteroaromatic, or a bicyclic fused-ring heteroaromatic group. Examples of said heteroaryl include, but are not limited to, pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl or triazolyl.

In this specification, unless stated otherwise, the term “halogeno” may be fluoro, chloro, bromo or iodo.

In this specification, unless stated otherwise, the term “C₁₋₆ fluoroalkyl” may be an alkyl substituted with one or more fluorine atoms. Examples of said fluoroalkyl include, but are not limited to, monofluoromethyl, trifluoromethyl, difluoromethyl and trifluoroethyl.

In another aspect of the invention there are provided compounds according to formula I wherein R¹ is aryl or heteroaryl each of which is optionally substituted with one or more of the following: —COOR³, —CONR³R⁴, —NHCOR³, or —NR³R⁴; R² is NO₂, NH₂, —NR⁵R⁶ or —NR⁶R⁷; R³ and R⁴ are each independently hydrogen or C₁₋₆alkyl or heterocycleC₀₋₆alkyl, and said C₁₋₆alkyl may be substituted with one or more B; or R³ and R⁴ form together a 5-, 6- or 7-membered heterocyclic ring containing 1 to 4 heteroatoms independently selected from N, O and S, and said ring may be substituted with one or more B; B is hydroxy, CN, R¹⁰, —COOR¹⁰, —NHCOR¹⁰, —NR¹⁰R¹¹, —CONR¹⁰R¹¹, or —OR¹⁰; R⁵ is phenyl or heteroaryl each of which is optionally substituted with one or more of —OR¹⁰, —R¹⁰, —CONR¹⁰R¹¹, —NR¹⁰R¹¹, or halogeno; R⁶ is hydrogen, or C₁₋₆alkyl; R⁷ is C₁₋₆alkyl; A is hydrogen, R⁸, or —NR⁸R⁹; R⁸ and R⁹ each independently are hydrogen, C₁₋₆alkyl; R¹⁰ and R¹¹ each independently are hydrogen, C₁₋₆alkyl, C₁₋₆alkanol, or; R¹⁰ and R¹¹ each independently are hydrogen, C₁₋₆alkyl, C₁₋₆fluoroalkyl or hydroxyC₁₋₆alkyl, or R¹⁰ and R¹¹ form together a 5-, 6- or 7-membered heterocyclic ring containing 1 to 4 heteroatoms independently selected from N, O and S, and said ring may be substituted with one or more B.

In another aspect of the invention there are provided compounds according to formula I wherein R¹ is phenyl optionally substituted with one or more of the following —OR³, —COOR³, —CONR³R⁴, —NHCOR³, —NR³R⁴, or —SO₂R³.

In one embodiment of this aspect of the invention there are provided compounds according to formula I wherein R³ and/or R⁴ are each independently hydrogen, C₁₋₆alkyl, or heterocycleC₀₋₆alkyl, and said C₁₋₆alkyl, may be substituted with one or more B; or R³ and R⁴ form together a 5-, 6- or 7-membered heterocyclic ring containing 1 to 4 heteroatoms independently selected from N, O and S, and said ring may be substituted with one or more B; B is CN, C₁₋₆alkyl, R¹⁰, —COOR¹⁰, —NHCOR¹⁰, —NR¹⁰R¹¹, —CONR¹⁰R¹¹, or —OR¹⁰.

In another aspect of the invention there are provided compounds according to formula I wherein R¹⁰ and R¹¹ each independently are hydrogen, C₁₋₆alkyl, C₁₋₆fluoroalkyl or hydroxyC₁₋₆alkyl, or R¹⁰ and R¹¹ form together a 5-, 6- or 7-membered heterocyclic ring containing 1 to 4 heteroatoms independently selected from N, O and S, and said ring may be substituted with one or more B.

In another aspect of the invention there are provided compounds according to formula I wherein R¹ is heteroaryl.

In another aspect of the invention there are provided compounds according to formula I wherein R² is NR⁵R⁶ and said R⁵ is phenyl optionally substituted with one or more (C₃₋₈cycloalkyl)C₀₋₆alkyl-, halogeno, and said R⁶ is hydrogen.

In one embodiment of this aspect of the invention there are provided compounds according to formula I wherein wherein said halogeno is chloro.

In another aspect of the invention there are provided compounds according to formula I wherein R² is NO₂, or NH₂.

In another aspect of the invention there are provided compounds according to formula I wherein A is hydrogen, R⁸, or NR⁸R⁹, and R⁸ and R⁹ each independently are hydrogen, or C₁₋₆alkyl, and said C₁₋₆alkyl may be substituted with one or more B; or R⁸ and R⁹ form together a 5-, 6- or 7-membered heterocyclic ring containing 1 to 4 heteroatoms independently selected from N, O and S, and said ring may be substituted with one or more B.

In another aspect of the invention there are provided compounds said compounds being:
(2-Chloro-phenyl)-(3-phenyl-1H-indazol-6-yl)-amine hydrochloride;
Phenyl-(3-phenyl-1H-indazol-6-yl)-amine hydrochloride;
(4-Fluoro-phenyl)-(3-phenyl-1H-indazol-6-yl)-amine hydrochloride;
(3-Phenyl-1H-indazol-6-yl)-(4-trifluoromethyl-phenyl)-amine hydrochloride;
(3-Phenyl-1H-indazol-6-yl)-(3-trifluoromethyl-phenyl)-anine hydrochloride;
(3-Phenyl-1H-indazol-6-yl)-pyridin-2-yl-amine hydrochloride;
Phenyl-[3-(1H-pyrrol-2-yl)-1H-indazol-6-yl]-amine hydrochloride;
(2-Methoxy-phenyl)-(3-phenyl-1H-indazol-6-yl)-amine;
(3-Phenyl-1H-indazol-6-yl)-pyridin-3-yl-amine hydrochloride;
Benzyl-(3-phenyl-1H-indazol-6-yl)-amine hydrochloride;
Cyclopropylmethyl-(3-phenyl-1H-indazol-6-yl)-amine hydrochloride;
Methyl-(3-phenyl-1H-indazol-6-yl)-amine hydrochloride;
6-Nitro-3-(1H-pyrrol-2-yl)-1H-indazole hydrochloride;
6-Nitro-3-pyridin-3-yl-1H-indazole hydrochloride;
3-Furan-2-yl-6-nitro-1H-indazole hydrochloride;
Dimethyl-[4-(6-nitro-1H-indazol-3-yl)-phenyl]-amine hydrochloride;
N-[3-(6-nitro-1H-indazol-3-yl)-phenyl]-acetamide;
3-Pyridin-3-yl-1H-indazol-6-ylamine;
3-(1H-Pyrrol-2-yl)-1H-indazol-6-ylamine hydrochloride;
3-(3-Methoxy-phenyl)-1H-indazol-6-ylamine hydrochloride;
N-(2-chlorophenyl)-3-[4-(methylsulfonyl)phenyl]-1H-indazol-6-amine hydrochloride;
Methyl 4 {6-[(2-chlorophenyl)amino]-1H-indazol-3-yl}benzoate dihydrochloride;
4-{6-[(2-Chlorophenyl)amino]-1H-indazol-3-yl}benzoic acid dihydrochloride;
Methyl 3-{6-[(2-chlorophenyl)amino]-1H-indazol-3-yl}benzoate dihydrochloride;
3-{6-[(2-Chlorophenyl)amino]-1H-indazol-3-yl}benzoic acid dihydrochloride;
N-(2-chlorophenyl)-3-{3-[(4-methylpiperazin-1-yl)carbonyl]phenyl}-1H-indazol-6-amine;
4-{6-[(2-Chlorophenyl)amino]-1H-indazol-3-yl}-N-[3-(4-methylpiperazin-1-yl)propyl]benzamide;
4-{6-[(2-Chlorophenyl)amino]-1H-indazol-3-yl}-N-(2-morpholin-4-ylethyl)benzamide;
4-{6-[(2-Cchlorophenyl)amino]-1H-indazol-3-yl}-N-[2-(dimethylamino)ethyl]benzamide;
4-{6-[(2-Chlorophenyl)amino]-1H-indazol-3-yl}-N-[3-(dimethylamino)propyl]benzamide;
4-{6-[(2-Chlorophenyl)amino]-1H-indazol-3-yl}-N-[3-carbamoylmethyl-benzamide;
N-(2-chlorophenyl)-3-[4(thiomorpholin-4-ylcarbonyl)phenyl]-1H-indazol-6-amine;
Methyl N-(4-{6-[(2-chlorophenyl)amino]-1H-indazol-3-yl}benzoyl)-N-methylglycinate;
1-(4-{6-[(2-Chlorophenyl)amino]-1H-indazol-3-yl} benzoyl)pyrrolidin-3-ol;
4-{6-[(2-Chlorophenyl)amino]-1H-indazol-3-yl}-N,N-bis(cyanomethyl)benzamide;
N-(2-Chlorophenyl)-3-(4-{[3-(dimethylamino)pyrrolidin-1-yl]carbonyl}phenyl)-1H-indazol-6-amine;
4-{6-[(2-Chlorophenyl)amino]-1H-indazol-3-yl}-N-[2-(dimethylamino)ethyl]-N-ethylbenzamide;
1-(4-{6-[(2-Chlorophenyl)amino]-1H-indazol-3-yl}benzoyl)piperidine-4-carboxamide;
4-{6-[(2-Chlorophenyl)amino]-1H-indazol-3-yl}-N-(2-hydroxyethyl)-N-methylbenzamide;
1-(4-{6-[(2-Chlorophenyl)amino]-1H-indazol-3-yl}benzoyl)piperidin-4-ol;
N-(2-chlorophenyl)-3-[4-(morpholin-4-ylcarbonyl)phenyl]-1H-indazol-6-amine;
3-{6-[(2-Chlorophenyl)amino]-1H-indazol-3-yl}-N-{3-[(2-hydroxyethyl)(methyl)amino]propyl}benzamide;
3-{6-[(2-Chlorophenyl)amino]-1H-indazol-3-yl}-N-(3-morpholin-4-ylpropyl)benzamide;
3-{6-[(2-Chlorophenyl)amino]-1H-indazol-3-yl}-N-[2-(diethylamino)-1-methylethyl]benzamide;
3-{6-[(2-Chlorophenyl)amino]-1H-indazol-3-yl}-N-[2-hydroxy-1-(hydroxymethyl)-1-methylethyl]benzamide;
3-{6-[(2-Chlorophenyl)amino]-1H-indazol-3-yl}-N-(2-morpholin-4-ylethyl)benzamide;
Ethyl 4-[(3-{6-[(2-chlorophenyl)amino]-1H-indazol-3-yl}benzoyl)amino]piperidine-1-carboxylate;
3-{6-[(2-Chlorophenyl)amino]-1H-indazol-3-yl)-N-(2-piperidin-1-ylethyl)benzamide;
3-{6-[(2-Chlorophenyl)amino]-1H-indazol-3-yl}-N-[2-(dimethylamino)ethyl]benzamide;
3-{6-[(2-Chlorophenyl)amino]-1H-indazol-3-yl}-N-[3-(dimethylamino)propyl]benzamide;
3-{6-[(2-Chlorophenyl)amino]-1H-indazol-3-yl}-N-(2-ethoxyethyl)benzamide;
3-{6-[(2-Chlorophenyl)amino]-1H-indazol-3-yl}-N-(2-hydroxyethyl)benzamide;
N-[2-(acetylamino)ethyl]-3-{6-[(2-chlorophenyl)amino]-1H-indazol-3-yl}benzamide;
3-{6-[(2-Chlorophenyl)amino]-1H-indazol-3-yl}-N-carbamoylmethyl-benzamide;
3-{6-[(2-Chlorophenyl)amino]-1H-indazol-3-yl}-N-(1-ethylpiperidin-3-yl)benzamide;
3-{6-[(2-Chlorophenyl)amino]-1H-indazol-3-yl}-N-(3-pyrrolidin-1-ylpropyl)benzamide;
3-{6-[(2-Chlorophenyl)amino]-1H-indazol-3-yl}-N-[3-(4-methylpiperazin-1-yl)propyl]benzamide;
3-{6-[(2-Chlorophenyl)amino]-1H-indazol-3-yl}-N-[(1-ethylpyrrolidin-2-yl)methyl]benzamide;
3-{6-[(2-Chlorophenyl)amino]-1H-indazol-3-yl}-N-(tetrahydrofuran-2-ylmethyl)benzamide;
(2-Chloro-phenyl)-(5-methyl-3-phenyl-1H-indazol-6-yl)-amine hydrochloride;
N-(2-morpholin-4-ylethyl)-6-nitro-3-phenyl-1H-indazol-5-amine hydrochloride;
(2-Fluoro-phenyl)-(3-phenyl-1H-indazol-6-yl)-amine hydrochloride;
3-(4-Methanesulfonyl-phenyl)-6-nitro-1H-indazole hydrochloride;
3-Furan-3-yl-6-nitro-1H-indazole hydrochloride;
   as a free base or a pharmaceutically acceptable salt thereof.

The present invention relates to the use of compounds of formula I as hereinbefore defined as well as to the salts thereof. Salts for use in pharmaceutical formulations will be pharmaceutically acceptable salts, but other salts may be useful in the production of the compounds of formula I. Such salts are possible, includes both pharmaceutically acceptable acid and base addition salts. A suitable pharmaceutically-acceptable salt of a compound of Formula I is, for example, an acid-addition salt of a compound of Formula I which is sufficiently basic, for example an acid-addition salt with an inorganic or organic acid such as hydrochloric.

Some compounds of formula I may have chiral centres and/or geometric isomeric centres (E- and Z-isomers), and it is to be understood that the invention encompasses all such optical, diastereoisomers and geometric isomers.

Certain compounds of the present invention may exist as tautomers. It is to be understood that the present invention encompasses all such tautomers.

The present invention relates to novel substituted indazole derivatives, which are inhibitors of c-Jun N-terminal kinases (JNKs). JNKs have been implicated in mediating a number of disorders. The invention relates to methods for producing these inhibitors. The invention also provides pharmaceutical compositions comprising the inhibitors of the invention and methods of utilizing these compositions in the treatment of various disorders.

Pharmaceutical Compositions

According to one aspect of the present invention there is provided a pharmaceutical composition comprising a compound of formula I, as a free base or a pharmaceutically acceptable salt, solvate or solvate of salt thereof, for use in the prevention and/or treatment of conditions associated with c-Jun N-terminal kinases (JNKs).

The composition may be in a form suitable for oral administration, for example as a tablet, for parenteral injection as a sterile solution or suspension. In general the above compositions may be prepared in a conventional manner using pharmaceutically carriers or diluents. Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

A compound of formula I, or a pharmaceutically acceptable salt, solvate or solvate of salt thereof, can be used on its own but will usually be administered in the form of a pharmaceutical composition in which the formula I compound/salt (active ingredient) is in association with a pharmaceutically acceptable diluent or carrier. Dependent on the mode of administration, the pharmaceutical composition may comprise from 0.05 to 99% w (percent by weight), for example from 0.10 to 50% w, of active ingredient, all percentages by weight being based on total composition.

A diluent or carrier includes water, aqueous polyethylene glycol, magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A composition of the invention can be in tablet or injectable form. The tablet may additionally comprise a disintegrant and/or may be coated (for example with an enteric coating or coated with a coating agent such as hydroxypropyl methylcellulose).

The invention further provides a process for the preparation of a pharmaceutical composition of the invention which comprises mixing a compound of formula I, or a pharmaceutically acceptable salt thereof, as hereinbefore defined, with a pharmaceutically acceptable diluent or carrier.

An example of a pharmaceutical composition of the invention is an injectable solution containing a compound of the invention, or a a pharmaceutically acceptable salt, solvate or solvate of salt thereof, as hereinbefore defined, and sterile water, and, if necessary, either sodium hydroxide or hydrochloric acid to bring the pH of the final composition to about pH 5, and optionally a surfactant to aid dissolution.

Liquid solution comprising a compound of formula I, or a salt thereof, dissolved in water.

| Solution | mg/mL |
|---|---|
| Active compound | 5.0% w/v |
| Pure water | To 100% |

Medical Use

The compounds of Formula I have activity as medicaments. In particular the compounds of formula I are potent JNK inhibitors and preferred compounds are selective JNK3 inhibitors. The present invention provides a compound of Formula I for use as a medicament. In particular the present invention provides a compound of Formula I for use in the prevention or treatment of conditions associated with JNK activation.

The present invention provides a method of treating or preventing conditions associated with JNK activation comprising the administration of a therapeutically effective amount of a compound of Formula I to a mammal (particularly a human including a patient) in need thereof.

In a further aspect the present invention provides the use of a compound of Formula I in the manufacture of a medicament for the treatment of conditions associated with JNK activation.

Conditions that may be treated by the compounds of this invention, according to Formula I, or a pharmaceutical composition containing the same, include any condition associated with JNK activation. Conditions associated with JNK activation include but are not limited to:
central or peripheral neurological degenerative disorders including Alzheimer's disease, cognitive disorders, Parkinson's disease, Huntington's disease, amyotrophic lateral sclerosis, Frontotemporal dementia Parkinson's Type, Parkinson dementia complex of Gaum, HIV dementia, corticobasal degeneration, dementia pugilistica, Down's syndrome, postencephelatic parkinsonism, progressive supranuclear palsy, Pick's Disease, Niemann-Pick's Disease, epilepsy, a peripheral neuropathy, spinal cord injury, head trauma; cancer, for example breast-, colorectal, pancreatic, prostate cancer.

In addition, JNK inhibitors of the instant invention may be capable of inhibiting the expression of inducible pro-inflammatory proteins. Therefore other conditions, which may be treated by the compounds of this invention, include edema, analgesia, fever and pain, such as neuromuscular pain, headache, cancer pain, dental pain and arthritis pain.

In the context of the present specification, the term “therapy” also includes “prevention” unless there are specific indications to the contrary. The terms “therapeutic” and “therapeutically” should be construed accordingly.

The term “condition”, unless stated otherwise, means any disorder and disease associated with JNK activity.

Non-Medical Use

In addition to their use in therapeutic medicine, the compounds of formula I or salt thereof, are also useful as pharmacological tools in the development and standardisation of in vitro and in vivo test systems for the evaluation of the effects of JNK inhibitor related activity in laboratory animals such as cats, dogs, rabbits, monkeys, rats and mice, as part of the search for new therapeutics agents.

Methods of Preparation

The compounds of this invention may be prepared by methods known to those skilled in the art for analogous compounds, as illustrated by the general schemes and procedures below and by the preparative examples that follow.

All starting materials are commercially available or prepared by methods known to those skilled in the art for analogous compounds, earlier described in the literature.

Unless specified otherwise, are R¹ to R¹¹, B and A defined as in formula I, X is halogen and PG is protecting group.
Synthetic Scheme Method 1:

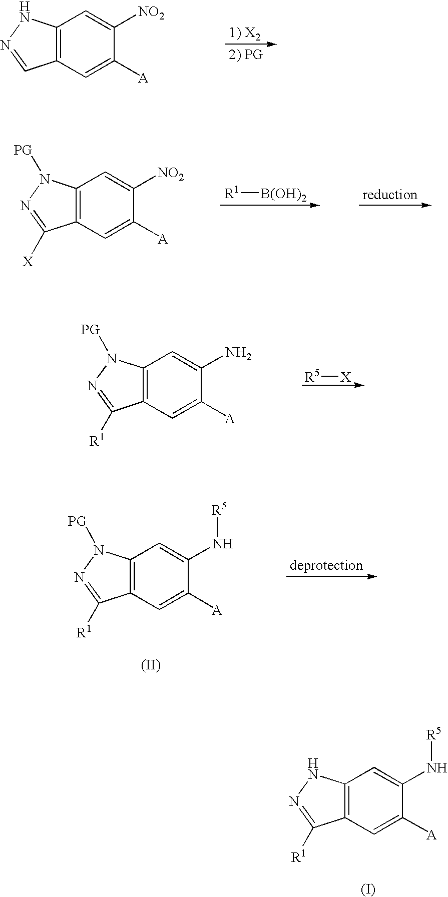

In the first step the indazole is halogenated by X₂, preferably iodine or bromine at room temperature. Then the indazole nitrogen is protected with PG, which represents an amino protecting group, for example t-butoxycarbonyl or 2-(trimethylsilyl)ethoxymethyl.

Methods of protecting and deprotecting amines are given in the standard text “Protecting groups in Organic Synthesis”, 2nd Edition (1991) by Greene and Wuts and also in the 3^{rd} edition of that book. Thereafter the R¹ group is introduced via their boronic acid derivatives R¹—B(OH)₂ using a palladium catalyzed reaction. This reaction can be performed in a solvent mixture such as toluene/ethanol at 80° C. in the presence of a palladium catalyst such as PdCl₂(dppf). The nitro group can then be reduced with catalytic hydrogenation using a catalyst such as platinum oxide or palladium on carbon under an atmosphere of hydrogen at room temperature. The R⁵ group is then introduced via their halide derivatives R⁵—X using a palladium catalyzed reaction to yield intermediate II. This reaction can be performed in an inert solvent such as toluene or tetrahydrofuran at elevated temperatures in the presence of a palladium catalyst such as Pd(OAc)₂ and (S)-BINAP or Pd(dba)₂ and DPPF together with a base such as cesium carbonate or sodium tert-butoxide.

Finally the amino protecting group is removed by hydrolysis, for example acid hydrolysis or treatment with tetrabutylammonium fluoride to yield compound I wherein R² is NR⁵R⁶.
Synthetic Scheme Method 1a:

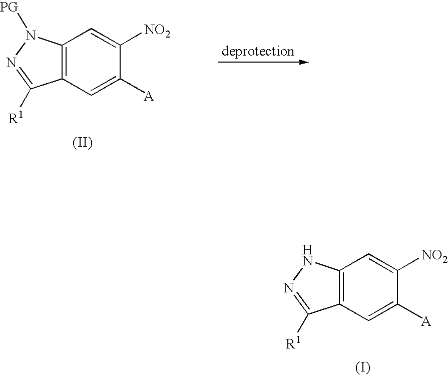

PG represents an amino protecting group, for example t-butoxycarbonyl or 2-(trimethylsilyl)ethoxymethyl. One method of deprotection of intermediate II is hydrolysis, for example acid hydrolysis or treatment with tetrabutylammonium fluoride to yield compound I wherein R² is NO₂.
Synthetic Scheme Method 1b:

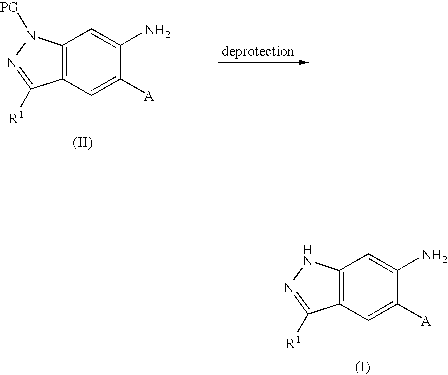

PG represents an amino protecting group, for example t-butoxycarbonyl or 2-(trimethylsilyl)ethoxymethyl. One method of deprotection of intermediate II is hydrolysis, for example acid hydrolysis or treatment with tetrabutylammonium fluoride to yield compound I wherein R² is NH₂.
Synthetic Scheme Method 2:

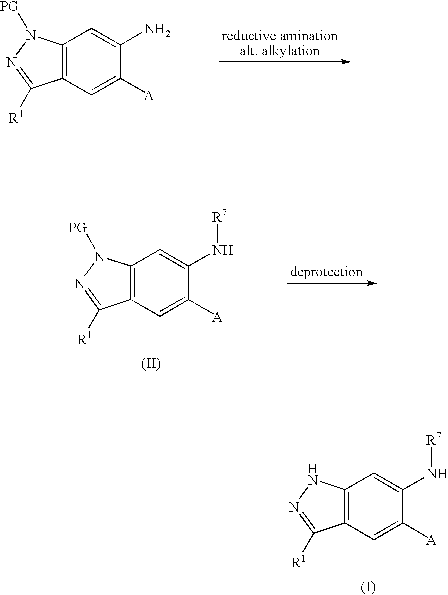

In the first step the amino group is alkylated. These alkylations may be performed by reacting the amine with an aldehyde in the presence of a reducing agent such as sodium triacetoxyborohydride or sodium cyanoborohydride, a reductive amination, or the amine can be reacted with an alkylhalide in the presence of a base such as potassium carbonate to yield intermediate II. In the second step the amino protecting group is removed for example by acid hydrolysis or treatment with tetrabutylammoniumfluoride to yield compound I wherein R² is NR⁶R⁷.
Synthetic Scheme Method 3:

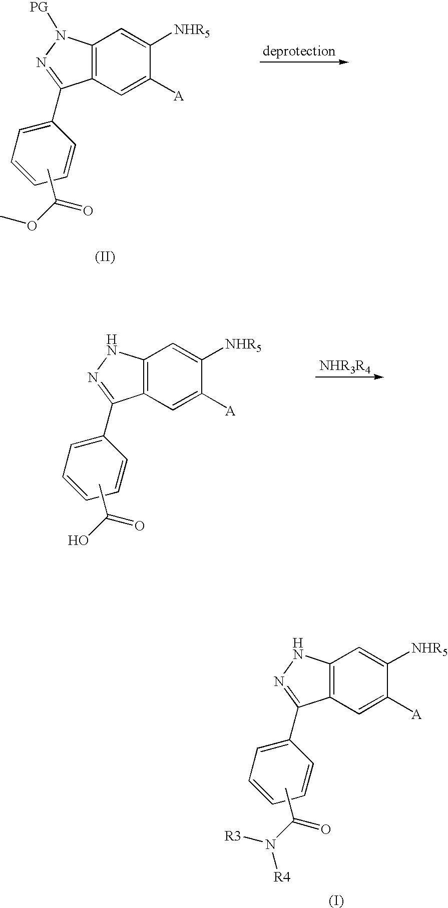

The amino protected indazole ester derivative II can be deprotected by acid hydrolysis, for example in aqueous HCl, to yield the intermediate carboxylic acids. These carboxylic acids can be converted to the amide compounds I. The amides I can be synthesized by reacting the acid with the corresponding amines in the presence of coupling reagents, such as 1,3-dicyclohexylcarbodiimide, 1-(3-dimethylaminopropyl)-3-ethylcarbodiimide, HATU or TBTU and HOBt, and a base, such as diisopropylethylamine, in an inert solvent, such as DMF, at 25° C. for a time of 1 h to 24 h.
Synthetic Scheme Method 4: 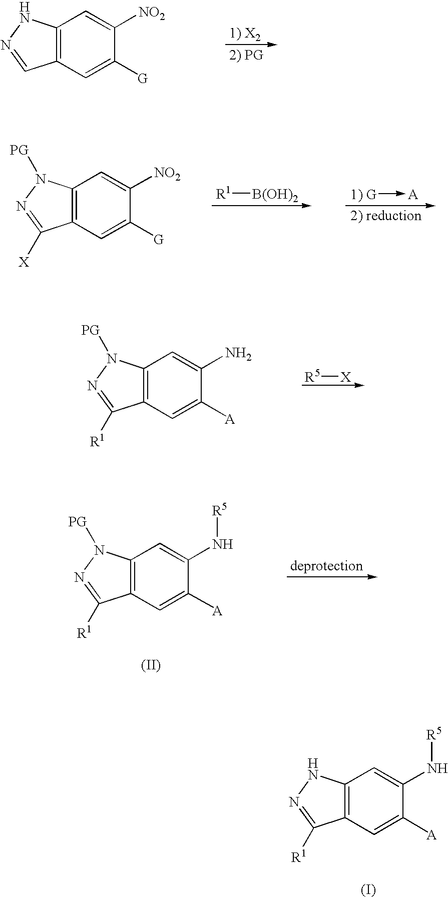
wherein G represents a group in A that can be converted to another group in A by general methods for those skilled in the art.

In the first step the indazole is halogenated by X₂, preferably iodine or bromine at room temperature. Then the indazole nitrogen is protected with PG, which represents an amino protecting group, for example t-butoxycarbonyl or 2-(trimethylsilyl)ethoxymethyl. Thereafter the R¹ group is introduced via their boronic acid derivatives R¹—B(OH)₂ using a palladium catalyzed reaction. This reaction can be performed in a solvent mixture such as is toluene/ethanol at 80° C. in the presence of a palladium catalyst such as PdCl₂(dppf). At this stage it is then possible to convert G into A. For example if G is halogen it can be displaced by an amine (HNR⁸R⁹) under catalytic conditions to yield the corresponding amino derivative, or the halogen can be reacted under aminocarbonylation or Heck arylation conditions. The nitro group can then be reduced with catalytic hydrogenation using a catalyst such as platinum oxide or palladium on carbon under an atmosphere of hydrogen at room temperature. The R⁵ group is then introduced via their halide derivatives R⁵—X using a palladium catalyzed reaction to yield II. This reaction can be performed in an inert solvent such as toluene or tetrahydrofuran at elevated temperatures in the presence of a palladium catalyst such as Pd(OAc)₂ and (S)-BINAP or Pd(dba)₂ and DPPF together with a base such as cesium carbonate or sodium tert-butoxide. Finally the amino protecting group is removed by hydrolysis, for example acid hydrolysis or treatment with tetrabutylammonium fluoride to yield I.
Synthetic Scheme Method 5: 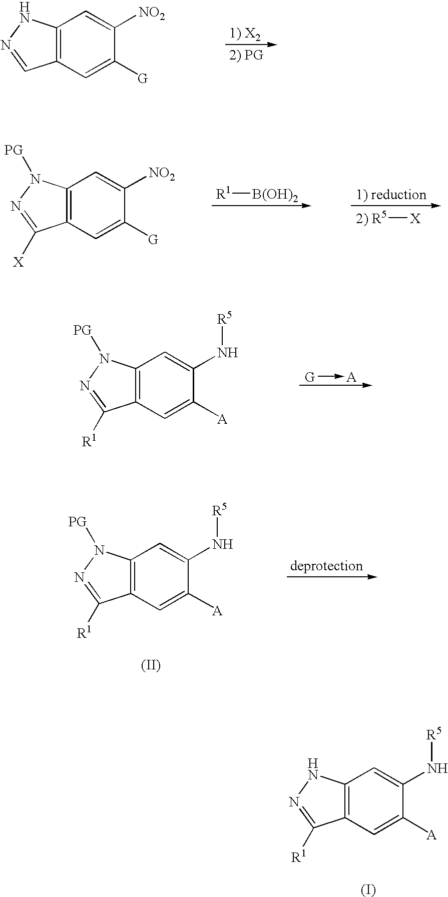
wherein G represents a group in A that can be converted to another group in A by general methods for those skilled in the art.

In the first step the indazole is halogenated by X₂, preferably iodine or bromine at room temperature. Then the indazole nitrogen is protected with PG, which represents an amino protecting group, for example t-butoxycarbonyl or 2-(trimethylsilyl)ethoxymethyl.

Thereafter the R¹ group is introduced via their boronic acid derivatives R¹—B(OH)₂ using a palladium catalyzed reaction. This reaction can be performed in a solvent mixture such as toluene/ethanol at 80° C. in the presence of a palladium catalyst such as PdCl₂(dppf). The nitro group can then be reduced with catalytic hydrogenation using a catalyst such as platinum oxide or palladium on carbon under an atmosphere of hydrogen at room temperature. The R⁵ group is then introduced via their halide derivatives R⁵—X using a palladium catalyzed reaction to yield II. This reaction can be performed in an inert solvent such as toluene or tetrahydrofuran at elevated temperatures in the presence of a palladium catalyst such as Pd(OAc)₂ and (S)-BINAP or Pd(dba)₂ and DPPF together with a base such as cesium carbonate or sodium tert-butoxide. At this stage it is then possible to convert G into A. For example if the G group is CN it can converted into a tetrazole, by treating the nitrile with an azide such as tributyltin azide or sodium azide. Another example could be if G is a methyl group it can be oxidized into a carboxylic acid moiety. Finally the amino protecting group is removed by hydrolysis, for example acid hydrolysis or treatment with tetrabutylammonium fluoride to yield I.
Synthetic Scheme Method 6:

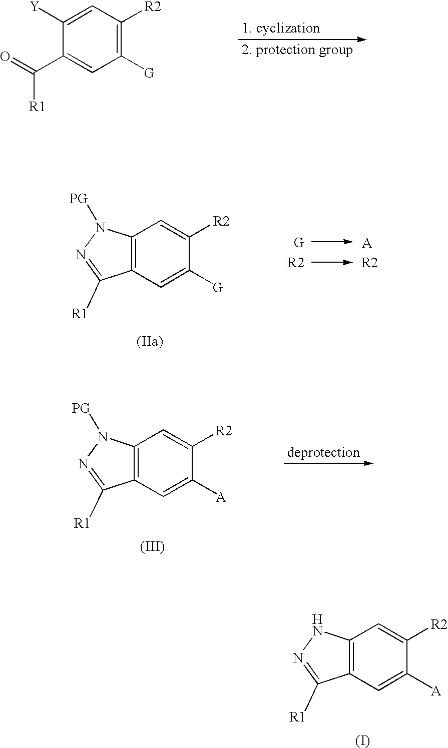

Intermediate IIa can be synthesized via cyclization of an aryl ketone substituted with G and R². If Y denotes a leaving group, such as F or Cl, the cyclization can be performed by heating the ketone in the presence of hydrazine. If Y is an amino group the cyclization can be performed by treating the starting material first with HNO₂ and then with a reducing agent, such as SnCl₂. After protection with a protection group, PG, G can be transformed into A by methods described in method 4 and 5 or by techniques well known to those skilled in the art of organic synthesis. Then R² is converted to NR⁵R⁶ as described in method 1 to yield intermediate II. Finally the protecting group is removed to yield compound I.

Another aspect the present invention provides a process for the preparation of a compound of Formula I comprising the de-protection of a compound of Formula II: 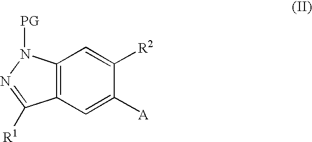
in which R¹, R² and A are as previously defined and PG represents an amino protecting group, for example t-butoxycarbonyl or 2-(trimethylsilyl)ethoxymethyl. Methods of protecting and deprotecting amines are given in the standard text “Protecting groups in Organic Synthesis”, 2nd Edition (1991) by Greene and Wuts and also in the 3^{rd} edition of that book. One method of deprotection is hydrolysis, for example acid or base hydrolysis.

Compounds of Formula II may be prepared as described in Method 1, 2, 4, 5 or 6.

Compounds of Formula II are novel, useful intermediates and are claimed as a further aspect of the present invention.

### WORKING EXAMPLES

The invention will now be described in more detail with the following examples that are not to be construed as limiting the invention.

All chemicals and reagents were used as received from suppliers. ¹H and ¹³C nuclear magnetic resonance (NMR) spectra were recorded on a BRUKER DPX 400 (400 MHz) spectrometer using the following solvents and references.
CDCl₃: ¹H NMR TMS (0.0 ppm) and ¹³C the central peak of CDCl₃ (77.0).
CD₃OD: ¹H NMR 3.31 ppm (central peak) and ¹³C 49.0 ppm (central peak).
DMSO-d₆: ¹H NMR 2.50 ppm (central peak) and ¹³C 39.51 ppm (central peak).
Mass spectra (TSP) were recorded on a Finigan MAT SSQ 7000 spectrometer.
Mass spectra (EI-DI) were recorded on a Finigan MAT SSQ 710 spectrometer.
LC-MS were recorded on a Waters Alliance 2790+ZMD spectrometer equipped with software Mass Lynx 3.5.

Flash column chromatography was carried out on silica gel 60 (230-400 mesh). Petroleum ether with boiling range 40-60° C. was used.

Ambient temperature is defined as a temperature between 16 and 25° C.

List of Abbreviations

| | |
|---|---|
| DMAP | Dimethylamino pyridine |
| DMF | N,N-Dimethylformamide |
| BINAP | 2,2′-Bis(diphenylphosphino)-1,1′-binaphthyl |
| Dppf | 1,1′-Bis(diphenylphosphino)ferrocene |
| EtOAc | Ethyl acetate |
| TEA | Triethyl amine |
| THF | Tetrahydrofuran |
| dba | Dibenzylideneacetone |
| OAc | Acetate |
| HATU | O-(7-Azabenzotriazol-1-yl)-N,N,N′,N′-tetramethyluronium |
| | hexafluorophosphate |
| TBTU | O-(1H-Benzotriazol-1-yl)-N,N,N′,N′-tetramethyluronium |
| | tetrafluoroborate |
| HOBt | 1-Hydroxybenzotriazole hydrate |

### Example 1

### (2-Chloro-phenyl)-(3-phenyl-1H-indazol-6-yl)-amine Hydrochloride

Method 1:

### (i) 3-Iodo-6-nitro-1H-indazole

Iodine (9.37 g, 18.4 mmol) and potassium hydroxide pellets (3.94 g, 69.0 mmol) were successively added into a DMF solution of 6-nitro-1H-indazole (3.00 g, 18.4 mmol) under stirring at ambient temperature. After 2.5 h, the reaction mixture was poured into 10% aqueous NaHSO₃ (150 mL) and extracted with dichloromethane (3×). The combined organic phases were washed with water and brine, dried (MgSO₄), filtered and the solvent evaporated. A small amount of dichloromethane was added and the solid was filtered, washed with dichloromethane and dried to give 4.36 g (82%) of the title compound as a yellow solid. ¹H NMR (CD₃OD): δ 7.61 (1H, d), 8.01 (1H, dd), 8.45 (1H, d). ¹³C NMR (CD₃OD): δ 87.8, 102.6, 111.2, 117.5, 125.6, 134.7, 142.8.

### (ii) 3-Iodo-6-nitro-indazole-1-carboxylic Acid Tert-Butyl Ester

To a solution of 3-iodo-6-nitro-1H-indazole (4.21 g, 14.6 mmol) in acetonitrile (100 mL) and methanol (50 mL) were added DMAP (0.185 g, 1.46 mmol), triethylamine (2.3 mL, 16.1 mmol) and di-tert-butyldicarbonate (3.82 g, 17.5 mmol) and the reaction was stirred at ambient temperature for 3.5 h. Water and dichloromethane were added, and the layers separated. The aqueous phase was extracted with dichloromethane (3×). The combined organic phases were washed (water, NaHCO₃ (sat. aq.), and brine), dried (MgSO₄), filtered and the solvent evaporated. Diethyl ether and a small amount of dichloromethane were added and the solid was filtered off, washed with diethyl ether and dried to give 4.89 g (86%) of the title compound as a pale yellow solid. ¹H NMR (CDCl₃): δ 1.69 (9H, s), 7.59 (1H, d), 8.17 (1H, dd), 9.01 (1H, d). ¹³C NMR (CDCl₃): δ 28.0, 87.0, 101.3, 111.1, 119.0, 122.9, 133.3, 138.7, 147.5, 148.9.

### (iii) 6-Nitro-3-phenyl-indazole-1-carboxylic Acid Tert-Butyl Ester

To a mixture of 3-iodo-6-nitro-indazole-1-carboxylic Acid Tert-Butyl Ester (3.30 g, 8.48 mmol) and PdCl₂(dppf) (0.312 g, 0.424 mmol) in toluene/ethanol 10/1 (100 mL), Na₂CO₃ (sat. aq.) (35 mL) was added followed by the addition of phenylboronic acid (1.14 g, 9.33 mmol). The reaction was stirred at 80° C. for 7 h under an atmosphere of nitrogen. Water and dichloromethane were added and the layers separated. The aqueous phase was extracted with dichloromethane (3×). The combined organic phases were washed with water and brine, dried (MgSO₄), filtered and the solvent evaporated. The crude material was purified by flash chromatography (petroleum ether/dichloromethane 50/50) to give 2.47 g (86%) of the title compound as a pale yellow solid. ¹H NMR (CDCl₃): δ 1.71 (9H, s), 7.48 (3H, m), 7.90 (2H, m), 8.03 (1H, d), 8.15 (1H, dd), 9.06 (1H, d). ¹³C NMR (CDCl₃): δ 28.1, 86.3, 111.3, 118.6, 122.2, 127.5, 128.2, 129.0, 129.9, 130.7, 140.1, 148.0, 148.4, 149.5.

### (iv) 6-Amino-3-phenyl-indazole-1-carboxylic Acid Tert-Butyl Ester

A mixture of 6-nitro-3-phenyl-indazole-1-carboxylic Acid Tert-Butyl Ester (1.00 g, 2.95 mmol) and platinum (IV) oxide (0.075 g, 0.30 mmol) in ethyl acetate/ethanol/tetrahydrofuran 1/1/1 (24 mL) was stirred under an atmosphere of hydrogen at ambient temperature for 4 h. The reaction mixture was filtered through a plug of celite and the solvents were evaporated to give 0.916 g (100%) of the title compound as a pale yellow solid. ¹H NMR (CDCl₃): δ 1.65 (9H, s), 3.98 (2H, br s), 6.64 (1H, dd), 7.40 (4H, m), 7.63 (1H, d), 7.88 (2H, m). ³C NMR (CDCl₃): δ 28.2, 84.4, 98.8, 114.0, 117.2, 122.3, 128.2, 128.7, 129.1, 132.2, 143.0, 147.6, 149.8(3), 149.8(5).

MS (TSP) nz/z-Boc-protecting group 210 (M+1).

### (v) 6-(2-Chloro-phenylamino)-3-phenyl-indazole-1-carboxylic Acid Tert-Butyl Ester

Pd(OAc)₂ (15.1 mg, 0.065 mmol) and (S)-BINAP (61.2 mg, 0.097 mmol) were premixed in dry tetrahydrofuran (3 mL) for 5 min. under an atmosphere of nitrogen. 1-Bromo-2-chlorobenzene (75 μL, 0.646 mmol) and the 6-amino-3-phenyl-indazole-1-carboxylic acid tert-butyl ester (199.8 mg, 0.646 mmol) were added followed by cesium carbonate (295.5 mg, 0.904 mmol). The reaction was stirred at 60° C. for 7 h under an atmosphere of nitrogen. Additional Pd(OAc)₂ (15.0 mg, 0.065 mmol), (S)-BINAP (61.4 mg, 0.097 mmol) and 1-bromo-2-chlorobenzene (75 μL, 0.646 mmol) were added and the reaction was stirred at 60° C. for 18 h under an atmosphere of nitrogen. Water and dichloromethane were added and the layers separated. The aqueous phase was extracted with dichloromethane (3×). The combined organic phases were washed with water and brine, dried (MgSO₄), filtered and the solvent evaporated. The crude material was purified by flash chromatography (petroleum ether/dichloromethane 30/70) to give 144.3 mg (53%) of the title compound as a white solid. ¹H NMR (CDCl₃): δ 1.69 (9H, s), 6.36 (1H, s), 6.92 (1H, m), 7.12 (1H, dd), 7.22 (1H, m), 7.42 (1H, dd), 7.50 (4H, m), 7.87 (1H, d), 7.94 (1H, s), 7.99 (2H, m). ¹³C NMR (CDCl₃): δ 28.2, 84.7, 102.3, 116.7, 117.6, 119.3, 122.0, 122.4, 123.1, 127.6, 128.2, 128.7, 129.2, 130.0, 132.0, 138.9, 142.4, 143.2, 149.5, 149.7.

MS (TSP) m/z-Boc-protecting group 320 and 322 (M+1).

### (vi) (2-Chloro-phenyl)-(3-phenyl-11H-indazol-6-yl)-amine Hydrochloride

To a solution of 6-(2-chloro-phenylamino)-3-phenyl-indazole-1-carboxylic acid tert-butyl easter (144.3 mg, 0.344 mmol) in methanol (2 mL) was added 4 M HCl in diethylether (1 mL) and the reaction was stirred at ambient temperature for 24 h. Additional 4 M HCl in diethyl ether (1 mL) was added and the reaction stirred for further 24 h at ambient temperature. The solvent was evaporated to give 117.1 mg (87%) of the title compound as a white solid. ¹H NMR (CD₃OD): δ 6.90 (1H, s), 7.07 (1H, t), 7.15 (1H, d), 7.26 (1H, t), 7.43 (2H, m), 7.58 (3H, m), 7.84 (3H, m). ¹³C NMR (CD₃OD): δ 91.8, 113.0, 119.4, 123.4, 123.5, 125.5, 126.7, 127.7, 128.1, 128.5, 130.1, 130.8, 131.7, 137.7, 142.0, 143.2, 149.0.

MS (TSP) m/z 320 and 322 (M+1).

### Example 2

### Phenyl-(3-phenyl-1H-indazol-6-yl)-amine Hydrochloride

### (i) 3-Phenyl-6-phenylamino-indazole-1-carboxylic Acid Tert-Butyl Ester

Prepared as described in method 1. Starting from 6-amino-3-phenyl-indazole-1-carboxylic acid tert-butyl ester (150.4 mg, 0.486 mmol) and bromobenzene (55 μL, 0.522 mmol) gave, after purification, 60.0 mg (32%) of the title compound as a pale yellow solid. ¹H NMR (CDCl₃): δ 1.58 (9H, s), 5.62 (1H, br s), 6.94 (2H, m), 7.15 (2H, m), 7.25 (2H, m), 7.39 (3H, m), 7.71 (1H, d), 7.75 (1H, s), 7.90 (2H, m). ¹³C NMR (CDCl₃): δ 28.2, 84.5, 99.7, 115.4, 118.2, 119.7, 122.3, 122.5, 128.1, 128.7, 129.1, 129.5, 132.1, 141.7, 142.6, 145.0, 149.5, 149.8.

MS (TSP) in/z-Boc-protecting group 286 (M+1).

### (ii) Phenyl-(3-phenyl-1H-indazol-6-yl)-amine Hydrochloride

To a solution of 3-phenyl-6-phenylamino-indazole-1-carboxylic Acid Tert-Butyl Ester (101.1 mg, 0.262 mmol) in methanol (2 mL) was added 4 M hydrochloric acid in diethyl ether (1 mL) and the reaction was stirred at ambient temperature for 48 h. The precipitate was filtered off, washed with diethyl ether and dried to give 45.0 mg of the title compound as a yellow solid. ¹H NMR (CD₃OD): δ 7.01 (1H, m), 7.08 (1H, m), 7.14 (1H, m), 7.25 (2H, m), 7.35 (2H, m), 7.61 (3H, m), 7.83 (3H, m). ¹³C NMR (CD₃OD): δ 112.5, 119.7, 121. 4(8), 121.5(3), 123.2, 124.1, 126.8, 128.5, 129.9, 130.1, 131.7, 140.9, 142.0, 143.7, 149.8.

MS (TSP) m/z 286 (M+1).

### Example 3

### (4-Fluoro-phenyl)-(3-phenyl-1H-indazol-6-yl)-anine Hydrochloride

### (i) 6-(4-Fluoro-phenylamino)-3-phenyl-indazole-1-carboxylic Acid Tert-Butyl Ester

Prepared as described in method 1. Starting from 6-amino-3-phenyl-indazole-1-carboxylic acid tert-butyl ester (300.4 mg, 0.971 mmol) and 4-bromo-fluorobenzene (110 μL, 0.999 mmol) afforded, after purification by flash chromatography (petroleum ether/dichloromethane 30/70), 74 mg (19%) of the title compound as a pale yellow solid.

¹H NMR (CDCl₃): δ 1.65 (9H, s), 6.04 (1H, br s), 6.91 (1H, dd), 7.04 (2H, m), 7.19 (2H, m), 7.47 (3H, m), 7.68 (1H, br s), 7.76 (1H, d), 7.96 (2H, m). MS (TSP) in/z-Boc-protecting group 304 (M+1).

### (ii) (4-Fluoro-phenyl)-(3-phenyl-1H-indazol-6-yl)-anine hydrochloride

To a solution of 6-(4-fluoro-phenylamino)-3-phenyl-indazole-1-carboxylic acid tert-butyl ester (74 mg, 0183 mmol) in methanol (1 mL) and tetrahydrofuran (1 mL) was added 4 M hydrochloric acid in diethyl ether (1 mL) and the reaction was stirred at ambient temperature for 3 days. The solvents were evaporated to obtain 66.3 mg of the title compound as a yellow solid. ¹H NMR (CD₃OD): δ 6.87 (1H, d), 7.03 (3H, m), 7.19 (2H, m), 7.57 (3H, m), 7.79 (3H, m). MS (TSP) m/z 304 (M+1).

### Example 4

### (3-Phenyl-1H-indazol-6-yl)-(4-trifluoromethyl-phenyl)-amine Hydrochloride

### (i) 3-Phenyl-6-(4-trifluoromethyl-phenylamino)-indazole-1-carboxylic Acid Tert-Butyl Ester

Prepared as described in method 1. Starting from 6-amino-3-phenyl-indazole-1-carboxylic acid tert-butyl ester (214.8 mg, 0.694 mmol) and 4-bromo-trifluoromethylbenzene (100 μL, 0.724 mmol) gave, after purification by flash chromatography (petroleum ether/dichloromethane 30/70), 249.1 mg (79%) of the title compound as a pale yellow solid. ¹H NMR (CDCl₃): δ 1.70 (9H, s), 6.25 (1H, s), 7.09 (1H, dd), 7.23 (2H, d), 7.52 (5H, m), 7.87 (1H, d), 7.98 (3H, m). ¹³C NMR (CDCl₃): δ 28.1, 84.8, 102.1, 116.8, 117.0, 119.3, 122.3, 122.7 (q), 124.4 (q), 126.6 (q), 128.1, 128.7, 129.2, 131.8, 142.3, 143.0, 145.5, 149.4, 149.8. MS (TSP) m/z-Boc-protecting group 354 (M+1).

### (ii) (3-Phenyl-1H-indazol-6-yl)-(4-trifluoromethyl-phenyl)-amine Hydrochloride

3-Phenyl-6-(4-trifluoromethyl-phenylamino)-indazole-1-carboxylic Acid Tert-Butyl Ester (135.9 mg, 0.300 mmol) was deprotected as described in method 1 to give 109.9 mg of the title compound as a yellow solid. ¹H NMR (CD₃OD): δ 7.22 (2H, m), 7.32 (2H, d), 7.54 (2H, d), 7.60 (3H, m), 7.83 (2H, m), 7.88 (1H, d). MS (TSP) m/z 354 (M+1).

### Example 5

### (3-Phenyl-1H-indazol-6-yl)-(3-trifluoromethyl-phenyl)-amine Hydrochloride

### (i) 3-Phenyl-6-(3-trifluoromethyl-phenylamino)-indazole-1-carboxylic Acid Tert-Butyl Ester

Prepared as described in method 1. Starting from 6-amino-3-phenyl-indazole-1-carboxylic acid tert-butyl ester (200.8 mg, 0.646 mmol) and 3-bromo-trifluoromethylbenzene (90 μL, 0.652 mmol) gave, after purification by flash chromatography (petroleum ether/dichloromethane 30/70), 113.1 mg (39%) of the title compound as a pale yellow solid. ¹H NMR (CDCl₃): δ 1.67 (9H, s), 6.23 (1H, s), 7.05 (1H, dd), 7.24 (1H, d), 7.45 (6H, m), 7.86 (2H, m), 7.98 (2H, m). MS (TSP) m/z-Boc-protecting group 354 (M+1).

### (ii) (3-Phenyl-1H-indazol-6-yl)-(3-trifluoromethyl-phenyl)-amine Hydrochloride

3-Phenyl-6-(3-trifluoromethyl-phenylamino)-indazole-1-carboxylic Acid Tert-Butyl Ester (113.1 mg, 0.249 mmol) was deprotected as described in method 1 to yield 87.8 mg of the is title compound as a yellow solid. ¹H NMR (CD₃OD): δ 7.07 (1H, m), 7.14 (1H, m), 7.24 (1H, d), 7.40 (3H, m), 7.55 (3H, m), 7.81 (3H, m). ¹³C NMR (CD₃OD): δ 91.5, 112.8, 116.6 (q), 118.8, 119.3, 119.5 (q), 123.3, 124.2 (q), 126.5, 128.4, 130.0, 130.3, 131.6, 131.9 (q), 141.7, 141.9, 143.0, 148.3. MS (TSP) nz/z 354 (M+1).

### Example 6

### (3-Phenyl-1H-indazol-6-yl)-pyridin-2-yl-amine Hydrochloride

### (i) 3-Phenyl-6-(pyridin-2-yl-amino)-indazole-1carboxylic Acid Tert-Butyl Ester

Prepared as described in method 1. Starting from 6-amino-3-phenyl-indazole-1-carboxylic acid tert-butyl ester (200.2 mg, 0.647 mmol) and 2-bromopyridine (65 μL×2, 0.668 mmol×2) gave, after purification by flash chromatography (petroleum ether/EtOAc 85/15), 168.8 mg (68%) of the title compound as a pale brown solid. ¹H NMR (CDCl₃ (Ref. 7.26 ppm)): δ 1.75 (9H, s), 6.84 (2H, m), 6.99 (1H, d), 7.23 (1H, m), 7.50 (3H, m), 7.58 (1H, m), 7.87 (1H, d), 8.00 (2H, m), 8.28 (1H, m), 8.46 (1H, d). LC-MS (API-ES) m/z 387 (M+1).

### (ii) (3-Phenyl-1H-indazol-6-yl)-pyridin-2-yl-amine

3-Phenyl-6-(pyridin-2-yl-amino)-indazole-1-carboxylic Acid Tert-Butyl Ester (168.8 mg, 0.437 mmol) was deprotected as described in method 1 and further purified by preparative HPLC to afford 71 mg of the title compound as a pale brown solid. ¹H NMR (CD₃OD): δ 6.75 (1H, m), 6.92 (1H, d), 7.09 (1H, dd), 7.37 (1H, m), 7.47 (2H, m), 7.53 (1H, m), 7.87 (4H, m), 8.13 (1H, m). ¹³C NMR (CD₃OD): δ 98.8, 111.1, 115.6, 116.7, 116.9, 121.9, 127.9, 128.5, 129.3, 134.1, 138.5, 140.7, 143.5, 145.6, 147.8, 156.6. MS (TSP) m/z 287 (M+1).

### Example 7

### Phenyl-[3-(1H-pyrrol-2-yl)-111-indazol-6-yl]-amine Hydrochloride

### (i) 3-(1-tert-Butoxycarbonyl-1H-pyrrol-2-yl)-6-nitro-indazole-1-carboxylic Acid Tert-Butyl Ester

Prepared as described in method 1. Starting from 3-iodo-6-nitro-indazole-1-carboxylic acid tert-butyl ester (1.25 g, 3.21 mmol) and (1-tert-butoxycarbonyl-1H-pyrrol-2-yl)-boronic acid (0.747 g, 3.54 mmol) gave, after purification by flash chromatography (petroleum ether/dichloromethane 40/60), 0.925 g (67%) of the title compound as a yellow solid. ¹H NMR (CDCl₃): δ 1.21 (9H, s), 1.68 (9H, s), 6.28 (1H, t), 6.56 (1H, dd), 7.43 (1H, dd), 7.62 (1H, d), 8.10 (1H, dd), 9.04 (1H, d). ¹³C NMR (CDCl₃): δ 27.4, 28.1, 84.5, 86.2, 111.2, 111.4, 118.2, 118.4, 122.1, 122.5, 124.3, 129.6, 139.1, 144.4, 147.9, 148.4(6), 148.4(8).

MS (EI-DI) m/z 428 (M)

### (ii) 6-Amino-3-(1-tert-butoxycarbonyl-1H-pyrrol-2-yl)-indazole-1-carboxylic Acid Tert-Butyl Ester

3-(1-tert-Butoxycarbonyl-1H-pyrrol-2-yl)-6-nitro-indazole-1-carboxylic acid tert-butyl ester (0.955 g, 2.23 mmol) was hydrogenated as described in method 1 to afford, after purification by flash chromatography (petroleum ether/dichloromethane 10/90), 0.646 g (73%) of the title compound as a pale yellow solid. ¹H NMR (CDCl₃): δ 1.21 (9H, s), 1.70 (9H, s), 4.25 (2H, br s), 6.31 (1H, t), 6.56 (1H, dd), 6.69 (1H, dd), 7.30 (1H, d), 7.47 (2H, m). ¹³C NMR (CDCl₃): δ 27.3, 28.2, 83.9, 84.3, 98.7, 111.0, 113.8, 117.2, 119.5, 122.1, 123.7, 124.0, 141.9, 144.9, 147.4, 148.9, 149.8. MS (TSP) m/z 399 (M+1).

### (iii) 3-(1-tert-Butoxycarbonyl-1H-pyrrol-2-yl)-6-phenylamino-indazole-1-carboxylic Acid Tert-Butyl Ester

Prepared as described in method 1. Starting from 6-amino-3-(1-tert-butoxycarbonyl-1H-pyrrol-2-yl)-indazole-1-carboxylic Acid Tert-Butyl Ester (199.8 mg, 0.501 mmol) and bromobenzene (55 μL, 0.522 mmol), gave, after purification by flash chromatography (petroleum ether/EtOAc 90/10), 96.1 mg (40%) of the title compound as a pale brown solid. ¹H NMR (CDCl₃): δ 1.23 (9H, s), 1.66 (9H, s), 6.31 (1H, t), 6.58 (2H, m), 7.01 (2H, m), 7.21 (2H, m), 7.36 (3H, m), 7.48 (1H, m), 7.83 (1H, s). LC-MS (API-ES) m/z 475.39 (M+1).

### (iv) Phenyl-[3-(1H-pyrrol-2-yl)-1H-indazol-6-yl]-amine Hydrochloride

3-(1-tert-Butoxycarbonyl-1H-pyrrol-2-yl)-6-phenylamino-indazole-1-carboxylic acid tert-butyl ester (96 mg, 0.202 mmol) was deprotected as described in method 1 to give 51 mg of the title compound as a dark green solid. ¹H NMR (CD₃OD): δ 6.36 (1H, m), 6.91 (1H, d), 7.05 (4H, m), 7.23 (2H, d), 7.33 (2H, t), 7.88 (1H, d). LC-MS (API-ES) m/z 275.27 (M+1).

### Example 8

### (2-Methoxy-phenyl)-(3-phenyl-1H1-indazol-6-yl)-amine

### (i) 6-(2-Methoxy-phenylamino)-3-phenyl-indazole-1-carboxylic Acid Tert-Butyl Ester

Prepared as described in method 1. Starting from 6-amino-3-phenyl-indazole-1-carboxylic acid tert-butyl ester (175.1 mg, 0.566 mmol) and 2-bromoanisole (70 μL×2, 0.566×2 mmol) gave, after purification by flash chromatography (petroleum ether/dichloromethane 10/90), 61 mg (26%) of the title compound as a pale yellow solid. ¹H NMR (CDCl₃ (Ref. 7.26 ppm)): δ 1.70 (9H, s), 3.91 (3H, s), 6.46 (1H, s), 6.95 (3H, m), 7.08 (1H, dd), 7.49 (4H, m), 7.82 (1H, d), 7.94 (1H, d), 8.00 (2H, m). ¹³C NMR (CDCl₃): δ 28.2, 55.6, 84.5, 100.2, 110.7, 116.1, 116.7, 118.4, 120.8, 121.5, 122.2, 128.2, 128.7, 129.1, 131.4, 132.2, 142.6, 144.4, 149.1, 149.5, 149.8. LC-MS (API-ES) m/z 416 (M+1).

### (ii) (2-Methoxy-phenyl)-(3-phenyl-1H-indazol-6-yl)-amine

6-(2-Methoxy-phenylamino)-3-phenyl-indazole-1-carboxylic Acid Tert-Butyl Ester (61 mg, 0147 mmol) was deprotected as described in method 1 and further purified by preparative HPLC to afford 31.2 mg of the title compound as a white solid. ¹H NMR (CDCl₃ (Ref. 7.26 ppm)): δ 3.92 (3H, s), 6.33 (1H, br s), 6.94 (3H, m), 6.99 (1H, dd), 7.19 (1H, br s), 7.41 (2H, m), 7.50 (2H, m), 7.89 (1H, d), 7.96 (2H, m). ¹³C NMR (CDCl₃): δ 55.6, 95.2, 110.7, 115.9, 116.0(3), 116.0(5), 120.8, 120.9, 121.9, 127.5, 128.1, 128.8, 132.1, 133.5, 142.6, 143.1, 145.6, 148.8. MS (TSP) nz/z 316 (M+1).

### Example 9

### (3-Phenyl-1H-indazol-6-yl)-pyridin-3-yl-amine Hydrochloride

### (i) 3-Phenyl-6-(pyridin-3-ylamino)-indazole-1-carboxylic Acid Tert-Butyl Ester

Prepared as described in method 1. Starting from 6-amino-3-phenyl-indazole-1-carboxylic acid tert-butyl ester (200.1 mg, 0.647 mmol) and 3-bromopyridine (65 μL×2, 0.647 mmol×2) gave, after purification by flash chromatography (petroleum ether/EtOAc 50/50), 49.7 mg (20%) of the title compound as a yellow-brownish solid. ¹H NMR (CDCl₃ (Ref. 7.26 ppm)): δ 1.68 (9H, s), 6.41 (1H, br s), 7.06 (1H, dd), 7.30 (1H, dd), 7.49 (3H, m), 7.66 (1H, m), 7.85 (1H, d), 7.90 (1H, s), 7.98 (2H, m) 8.25 (1H, m), 8.58 (1H, d). MS (TSP) nz/z-Boc-protecting group 287 (M+1).

### (ii) (3-Phenyl-1H-indazol-6-yl)-pyridin-3-yl-amine Hydrochloride

To a solution of 3-phenyl-6-(pyridin-3-ylamino)-indazole-1-carboxylic Acid Tert-Butyl Ester (46.0 mg, 0.119 mmol) in methanol (1.5 mL) was added 4 M hydrochloric acid in diethyl ether (1 mL) and the reaction stirred for 17 h at ambient temperature. Additional 4 M hydrochloric acid in diethyl ether (1 mL) was added and the reaction stirred for 9 h at ambient temperature. The precipitate formed was filtered off, washed with diethyl ether and dried to give 33.2 mg of the title compound as a yellow solid. ¹H NMR (CD₃OD): δ 7.29 (1H, dd), 7.52 (1H, d), 7.58 (3H, m), 7.84 (3H, m), 8.01 (1H, d), 8.14 (1H, d), 8.34 (1H, dd), 8.68 (1H, d). ¹³C NMR (CD₃OD): δ 98.1, 115.9, 119.4, 124.2, 128.2, 128.4, 128.5, 129.0, 130.1, 131.3, 131.8, 132.2, 142.7, 143.1, 143.4, 144.1. LC-MS (API-ES) m/z 287 (M+1).

### Example 10

### Benzyl-(3-phenyl-1H-indazol-6-yl)-amine Hydrochloride

Method 2:

### (i) 6-Benzylamino-3-phenyl-indazole-1-carboxylic Acid Tert-Butyl Ester

6-Amino-3-phenyl-indazole-1-carboxylic Acid Tert-Butyl Ester (174.7 mg, 0.566 mmol) and benzaldehyde (60 μL, 0.566 mmol) were mixed in 1,2-dichloroethane (5 mL) and then treated with sodium triacetoxyborohydride (177.4 mg, 0.792 mmol) and acetic acid (35 μL, 0.566 mmol). The mixture was stirred at ambient temperature for 5 h under an atmosphere of nitrogen. The reaction mixture was quenched by adding aqueous 1 N NaOH, and the layers separated. The aqueous phase was extracted with dichloromethane (3×). The combined organic phases were washed with water and brine, dried (MgSO₄), filtered and the solvent evaporated. The crude material was purified by flash chromatography (petroleum ether/dichloromethane 20/80) to give 204.0 mg (90%) of the title compound as a pale yellow solid. ¹H NMR (CDCl₃): δ 1.68 (9H, s), 4.45 (2H, s), 6.72 (1H, d), 7.32 (1H, m), 7.38 (5H, m), 7.48 (3H, m), 7.72 (1H, d), 7.96 (2H, m). MS (TSP) m/z-Boc-protecting group 300 (M+1).

### (ii) Benzyl-(3-phenyl-1H-indazol-6-yl)-amine Hydrochloride

To a solution of 6-benzylamino-3-phenyl-indazole-1-carboxylic Acid Tert-Butyl Ester (99.7 mg, 0.250 mmol) in methanol (3 mL) was added 4 M hydrochloric acid in diethyl ether and the reaction stirred for 48 h at ambient temperature. The solvent was evaporated to give 60.5 mg of the title compound as a pale brown solid. ¹H NMR (CD₃OD): δ 4.47 (2H, s), 6.97 (1H, d), 7.31 (6H, m), 7.51 (3H, m), 7.80 (3H, m). MS (TSP) m/z 300 (M+1).

### Example 11

### Cyclopropylmethyl-(3-phenyl-1H-indazol-6-yl)-amine Hydrochloride

### (i) 6-(Cyclopropylmethyl-amino)-3-phenyl-indazole-1-carboxylic Acid Tert-Butyl Ester

Prepared as described in method 2. Starting from 6-amino-3-phenyl-indazole-1-carboxylic acid tert-butyl ester (155.9 mg, 0.504 mmol) and cyclopropanecarboxaldehyde (40 μL, 0.535 mmol) gave, after purification by flash chromatography (petroleum ether/EtOAc 90/10), 134.3 mg (73%) of the title compound as a white solid. ¹H NMR (CDCl₃ (ref 7.26 ppm)): δ 0.29 (2H, q), 0.59 (2H, q), 1.16 (11H, m), 1.73 (9H, s), 3.07 (2H, d), 4.26 (1H, br s), 6.67 (1H, dd), 7.30 (1H, s), 7.46 (3H, m), 7.69 (1H, d), 7.96 (2H, m). ¹³C NMR (CDCl₃): δ 3.51, 10.6, 28.2, 48.9, 84.1, 94.8, 113.3, 116.0, 121.9, 128.1, 128.6, 129.0, 132.4, 143.5, 149.5, 149.8, 149.9. LC-MS (API-ES) m/z 364 (M+1).

### (ii) Cyclopropylmethyl-(3-phenyl-1H-indazol-6-yl)-amine Hydrochloride

To a solution of 6-(cyclopropylmethyl-amino)-3-phenyl-indazole-1carboxylic acid tert-butyl ester (134.3 mg, 0.370 mmol) in methanol (2 mL) was added 4 M hydrochloric acid in diethyl ether (1 mL) and the reaction stirred for 24 h at ambient temperature. Additional 4 M hydrochloric acid in diethyl ether (1 mL) was added and the reaction stirred for 7 h at ambient temperature. The white precipitate formed was filtered off, washed with diethyl ether and dried to give 92.5 mg of the title compound as a white solid. ¹H NMR (DMSO): δ 0.40 (2H, m), 0.54 (2H, m), 1.14 (1H, m), 3.26 (2H, d), 7.41 (2H, m), 7.53 (2H, m), 7.89 (1H, br s), 7.99 (2H, d), 8.20 (1H, d). ¹³C NMR (DMSO): 34.17, 7.26, 55.6, 105.3, 116.5, 119.3, 122.3, 126.9, 128.1, 129.0, 133.0, 135.1, 141.2, 143.4. MS (TSP) m/z 264 (M+1).

### Example 12

### Methyl-(3-phenyl-1H-indazol-6-yl)-amine Hydrochloride

### (i) 6-Methylamino-3-phenyl-indazole-1-carboxylic Acid Tert-Butyl Ester

Prepared as described in method 2. Starting from 6-amino-3-phenyl-indazole-1-carboxylic acid tert-butyl ester (175.4 mg, 0.567 mmol) and formaldehyde (37% aq. sol.) (45 μL, 0.600 mmol) gave after purification by flash chromatography (petroleum ether/EtOAc 80/20), 71.9 mg (39%) of the title compound as a white solid. ¹H NMR (CDCl₃): a 1.65 (9H, s), 2.84 (3H, s), 4.17 (1H, br s), 6.56 (1H, dd), 7.21 (1H, s), 7.38 (3H, m), 7.59 (1H, d), 7.88 (2H, m). ¹³C NMR (CDCl₃): δ 28.2, 30.5, 84.1, 94.3, 113.2, 116.0, 121.8, 128.1, 128.6, 128.9, 132.4, 143.5, 149.8, 149.9, 150.4. LC-MS (API-ES) m/z 324 (M+1).

### (ii) Methyl-(3-phenyl-1H-indazol-6-yl)-amine Hydrochloride

6-Methylamino-3-phenyl-indazole-1-carboxylic Acid Tert-Butyl Ester (71.9 mg, 0.222 mmol) was deprotected as described in method 2 to give, after purification, 39.2 mg of the title compound as a pale pink solid. ¹H NMR (DMSO): δ 2.96 (3H, s), 7.31 (1H, m), 7.43 (1H, m), 7.53 (2H, m), 7.74 (1H, br s), 7.99 (2H, m), 8.18 (1H, d). MS (TSP) m/z 224 (M+1).

### Example 13

### 6-Nitro-3-(1H-pyrrol-2-yl)-1H-indazole Hydrochloride

Method 1a:

To a solution of 3-(1-tert-butoxycarbonyl-1H-pyrrol-2-yl)-6-nitro-indazole-1-carboxylic acid tert-butyl ester (148.1 mg, 0.345 mmol) in methanol (2 mL) and tetrahydrofuran (1 mL) was added 4 M hydrochloric acid in diethyl ether (1 mL) and the reaction was stirred at ambient temperature for 48 h. Additional 4 M hydrochloric acid in diethyl ether (1 mL) was added and the reaction was stirred at ambient temperature for 24 h. The precipitate formed was filtered off, washed with diethyl ether and dried to afford 55 mg of the title compound as a yellow-greenish solid. ¹H NMR (DMSO): δ 6.21 (1H, m), 6.81 (1H, m), 6.90 (1H, m), 7.95 (1H, dd), 8.27 (1H, d), 8.43 (1H, d). ¹³C NMR (DMSO): δ 107.1, 107.6, 108.9, 114.7, 119.7, 122.1, 122.2, 123.7, 138.9, 139.8, 146.0. MS (EI-DI) in/z 228 (M).

### Example 14

### 6-Nitro-3-pyridin-3-yl-1H-indazole Hydrochloride

### (i) 6-Nitro-3-pyridin-3-yl-indazole-1-carboxylic Acid Tert-Butyl Ester

Prepared according to method 1. Starting from 3-iodo-6-nitro-indazole-1-carboxylic acid tert-butyl ester (199.8 mg, 0.513 mmol) and pyridine-3-boronic acid (76.2 mg, 0.620 mmol) afforded, after purification by flash chromatography (n-heptane/EtOAc 50/50), 46.5 mg (27%) of the title compound as a pale yellow solid. ¹H NMR (CDCl₃): δ 1.80 (9H, s), 7.53 (1H, br s), 8.10 (1H, d), 8.29 (1H, d), 8.35 (1H, d), 8.79 (1H, br s), 9.18 (1H, s), 9.25 (1H, br s). LC-MS (API-ES) m/z 341.24 (M+1).

### (ii) 6-Nitro-3-pyridin-3-yl-1H-indazole Hydrochloride

6-Nitro-3-pyridin-3-yl-indazole-1-carboxylic Acid Tert-Butyl Ester (46.5 mg, 0.137 mmol) was deprotected as described in method 1a to afford 37 mg of the title compound as a yellow solid. ¹H NMR (CD₃OD): δ 8.18 (1H, d), 8.24 (1H, t), 8.30 (1H, d), 8.59 (1H, d), 8.84 (1H, d), 9.23 (1H, d), 9.47 (1H, s). ¹³C NMR (CD₃OD): δ 108.6, 117.8, 121.3, 123.8, 128.6, 134.4, 138.4, 139.7, 140.6, 141.7, 144.0, 147.5.

### Example 15

### 3-Furan-2-yl-6-nitro-IL-indazole Hydrochloride

### (i) 3-Furan-2-yl-6-nitro-indazole-1-carboxylic Acid Tert-Butyl Ester

Prepared as described in method 1. Starting from 3-iodo-6-nitro-indazole-1-carboxylic acid tert-butyl ester (199 mg, 0.511 mmol) and furan-2-boronic acid (63.5 mg, 0.568 mmol) afforded, after flash chromatography (petroleum ether/dichlorometyhane 40/60), 136 mg (81%) of the title compound as a yellow solid. ¹H NMR (CDCl₃): δ 1.71 (9H, s), 6.56 (1H, dd), 7.16 (1H, d), 7.61 (1H, d), 8.17 (1H, dd), 8.29 (1H, d), 9.01 (1H, d). ¹³C NMR (CDCl₃): δ 28.1, 86.6, 110.8, 111.1, 112.0, 118.8, 123.1, 126.5, 139.6, 141.2, 144.1, 146.7, 148.2, 148.3. LC-MS (API-ES) m/z 330 (M+1).

### (ii) 3-Furan-2-yl-6-nitro-1H-indazole Hydrochloride

To a solution of 3-furan-2-yl-6-nitro-indazole-1-carboxylic Acid Tert-Butyl Ester in is methanol (1 mL) and THF (2 mL) was added 4 M HCl in diethyl ether (1 mL) and the reaction stirred for 24 h. Additional 4 M HCl in diethyl ether (1 mL) was added and the reaction stirred for 24 h. Starting material left according to TLC, so the solvent were evaporated and THF (2 mL) was added followed by 4 M HCl in diethyl ether (1 mL) and the reaction stirred for 65 h. The solvents were evaporated. A small amount of methanol and diethyl ether were added and the solid was filtered, washed with diethyl ether and dried to afford 25 mg of the title compound as a yellow solid. ¹H NMR (DMSO): δ 6.72 (1H, dd), 7.12 (1H, d), 7.90 (1H, d), 8.02 (1H, dd), 8.30 (1H, d), 8.48 (1H, d). ¹³C NMR (DMSO): δ 107.4, 107.7, 111.9, 115.6, 122.0, 122.2, 136.6, 139.5, 143.4, 146.2, 147.5.

LC-MS (API-ES) m/z 230 (M+1).

### Example 16

### Dimethyl-[4-(6-nitro-1H-indazol-3-yl)-phenyl]-amine Hydrochloride

### (i) 3-(4-Dimethylamino-phenyl)-6-nitro-indazole-1-carboxylic Acid Tert-Butyl Ester

To a mixture of 3-iodo-6-nitro-indazole-1-carboxylic Acid Tert-Butyl Ester (199.8 mg, 0.514 mmol) and PdCl₂(dppf) (20.0 mg, 0.026 mmol) in toluene/ethanol 10/1 (6 mL), Na₂CO₃ (sat. aq.) (2 mL) was added followed by the addition of 4-(dimethylamino)phenylboronic acid (93.8 mg, 0.565 mmol). The reaction was stirred at 80 IC for 7 h under an atmosphere of nitrogen. Starting material still remained according to TLC, so additional PdCl₂(dppf) (20.0 mg, 0.026 mmol) was added and the reaction stirred at 80° C. for 18 h under an atmosphere of nitrogen. The reaction was still incompleted, so additional PdCl₂(dppf) (20.0 mg, 0.026 mmol) and 4-(dimethylamino)phenylboronic acid (46.2 mg, 0.280 mmol) were added and the reaction was stirred at 80° C. for 4 h under an atmosphere of nitrogen. Water and dichloromethane were added and the layers separated. The aqueous phase was extracted with dichloromethane (3×). The combined organic phases were washed with water and brine, dried (MgSO₄), filtered and the solvent evaporated. The crude material was purified by flash chromatography (petroleum ether/dichloromethane 10/90) to give 136.3 mg (69%) of the title compound as a yellow solid. ¹H NMR (CDCl₃): δ 1.78 (9H, s), 3.05 (6H, s), 6.82 (2H, d), 7.87 (2H, m), 8.10 (1H, d), 8.19 (1H, dd), 9.08 (1H, d). ¹³C NMR (CDCl₃): δ 28.1, 40.2, 85.8, 111.1, 112.1, 118.2, 118.3, 122.4, 127.8, 129.1, 140.1, 147.8, 148.6, 149.8, 151.3. LC-MS (API-ES) nz/z 383 (M+1).

### (ii) Dimethyl-[4-(6-nitro-1H-indazol-3-yl)-phenyl]-amine Hydrochloride

To a solution of 3-(4-dimethylamino-phenyl)-6-nitro-indazole-1-carboxylic acid tert-butyl ester (136.3 mg, 0.356 mmol) in methanol (2 mL) was added 4 M hydrochloric acid in diethyl ether (1 mL) and the reaction stirred for 24 h at ambient temperature. Additional 4 M hydrochloric acid in diethyl ether (1 mL) was added and the reaction stirred for 24 h at ambient temperature. The white precipitate formed was filtered off, washed with diethyl ether and dried to give 98.1 mg of the title compound as a yellow solid. LC-MS (API-ES) m/z 283 (M+1). To get conclusive NMR spectra, the free amine was generated by extracting the HCl salt (in dichloromethane) with Na₂CO₃ (sat. aq.). The layers were then separated and the dichloromethane layer was evaporated to obtain the free amine. ¹H NMR (CDCl₃): δ 3.00 (6H, s), 6.83 (2H, d), 7.78 (2H, d), 7.96 (1H, dd), 8.04 (1H, d), 8.13 (1H, d). ¹³C NMR (CDCl₃): δ 40.4, 107.0, 112.6, 115.6, 119.6, 122.3, 124.1, 128.6, 140.3, 146.8, 146.9, 150.9.

### Example 17

### N-[3-(6-Nitro-1H-indazol-3-yl)-phenyl]-acetamide

### (i) N-[3-(6-Nitro-1H-indazol-3-yl)-phenyl]-acetamide

To a mixture of 3-iodo-6-nitro-indazole-1-carboxylic Acid Tert-Butyl Ester (200.2 mg, 0.514 mmol) and PdCl₂(dppf) (20 mg, 0.027 mmol) in DMF (4 mL), Na₂CO₃ (sat. aq.) (2 mL) was added followed by the addition of (3-acetylaminophenyl)boronic acid (110.4 mg, 0.617 mmol). The reaction was stirred at 80° C. for 7 h under an atmosphere of nitrogen.

Water and dichloromethane were added and the layers separated. The aqueous phase was extracted with dichloromethane (3×). The combined organic phases were washed with water and brine, dried (MgSO₄), filtered and the solvent evaporated. According to LC-MS the unprotected compound (LC-MS (API-ES) m/z 383 (M+1)) was formed but not the protected. The crude material was purified by flash chromatography (petroleum ether/EtOAc 50/50 then 25/75) to obtain the title compound as a pale yellow solid in low yield (26 mg). The material was further purified by preparative HPLC to give 9 mg of the title compound as a yellow solid. ¹H NMR (DMSO): δ 2.09 (3H, s), 7.46 (1H, t), 7.67 (2H, m), 8.03 (1H, dd), 8.26 (1H, d), 8.32 (1H, s), 8.51 (1H, s). ¹³C NMR (DMSO): δ 24.1, 107.7, 115.4, 117.3, 118.7, 121.4, 121.8, 123.0, 129.5, 132.8, 140.0, 140.5, 143.8, 145.7, 168.5. LC-MS (API-ES) m/z 297.17 (M+1).

### Example 18

### 3-Pyridin-3-yl-111-indazol-6-ylamine

A mixture of 6-nitro-3-pyridin-3-yl-1H-indazole (hydrochloride) (37 mg, 0.118 mmol) and platinum (IV) oxide (3 mg, 0.012 mmol) in methanol (4.5 mL) was stirred under an atmosphere of hydrogen at ambient temperature for 6.5 h. The reaction mixture was filtered and the solvent evaporated. The crude material was purified by preparative HPLC to obtain 8 mg of the title compound as a pale yellow solid. ¹H NMR (CD₃OD): δ 6.81 (1H, m), 6.89 (1H, s), 7.59 (1H, m), 7.78 (1H, d), 8.38 (1H, d), 8.57 (1H, d), 9.12 (1H, s).

MS (TSP) m/z 211 (M+1).

### Example 19

### 3-(1H-Pyrrol-2-yl)-1H-indazol-6-ylamine Hydrochloride

6-Amino-3-(1-tert-butoxycarbonyl-1H-pyrrol-2-yl)-indazole-1-carboxylic acid tert-butyl ester (74.9 mg, 0.188 mmol), prepared according to method 1, was deprotected to afford 44.1 mg of the title compound as a green-brownish solid. ¹H NMR (DMSO): δ 6.19 (1H, m), 6.75 (1H, m), 6.86 (1H, m), 7.12 (1H, dd), 7.60 (1H, s), 8.13 (1H, d). ¹³C NMR (DMSO): δ 104.8, 107.3, 108.8, 116.0, 118.5, 119.3, 122.4, 124.3, 130.5, 138.5, 140.9. MS (TSP) m/z 199 (M+1).

### Example 20

### 3-(3-Methoxy-phenyl)-1H-indazol-6-ylamine Hydrochloride

### (i) 6-Amino-3-(3-methoxy-phenyl)-indazole-1-carboxylic Acid Tert-Butyl Ester

3-(3-Methoxy-phenyl)-6-nitro-indazole-1-carboxylic Acid Tert-Butyl Ester (94 mg, 0.252 mmol), prepared according to method 1, was hydrogenated to give, after purification by flash chromatography (n-heptane/EtOAc 70/30), 84 mg (98%) of the title compound as a pale yellow solid. ¹H NMR (CDCl₃): δ 1.73 (9H, s), 3.89 (3H, s), 4.43 (2H, br s), 6.76 (1H, m), 6.99 (1H, m), 7.40 (1H, t), 7.51 (3H, m), 7.74 (1H, d). ¹³C NMR (CDCl₃): δ 28.1, 55.3, 84.4, 98.6, 113.2, 113.9, 115.1, 117.0, 120.6, 122.3, 129.6, 133.5, 143.0, 147.9, 149.7, 149.8, 159.7. MS (TSP) m/z-Boc-protecting group 240 (M+1).

### (ii) 3-(3-Methoxy-phenyl)-1H-indazol-6-ylamine Hydrochloride

6-Amino-3-(3-methoxy-phenyl)-indazole-1-carboxylic Acid Tert-Butyl Ester (84 mg, 0.248 mmol) was deprotected with HCl to afford 50 mg of the title compound as a pale brown solid. ¹H NMR (DMSO): δ 3.85 (3H, s), 7.00 (1H, d), 7.10 (1H, d), 7.45 (2H, m), 7.55 (2H, m), 8.12 (1H, d). ¹³C NMR (DMSO): δ 55.1, 103.7, 111.8, 113.8, 116.4, 118.6, 119.2, 122.1, 130.1, 132.0, 134.5, 141.5, 143.2, 159.6. MS (TSP) m/z 240 (M+1).

### Example 21

### N-(2-Chlorophenyl)-3-[4-(methylsulfonyl)phenyl]-1H-indazol-6-amine Hydrochloride

Prepared according to method 1. Starting from 3-iodo-6-nitro-indazole-1-carboxylic acid tert-butyl ester and 4-(methanesulfonyl)phenylboronic acid to afford 3-(4-methanesulfonyl-phenyl)-6-nitro-indazole-1-carboxylic Acid Tert-Butyl Ester. The nitro group was reduced and reacted with 1-bromo-2-chlorobenzene to yield after deprotection 60 mg of the title compound as an off-white solid. ¹H NMR (CD₃OD) 3 ppm 3.10 (s, 3H), 6.97 (m, 1H), 7.07 (m, 1H), 7.19 (m, 1H), 7.35 (m, 2H), 7.88 (m, 1H) 8.08 (m, 4H). ¹³C NMR(CD₃OD): 343.7, 116.5, 119.4, 123.5, 123.7, 125.6, 128.0, 129.4, 129.9, 130.1, 132.0, 138.2, 141.1, 142.9, 143.5, 145.3, 147.7. MS (TSP) m/z 398 (M+1).

### Example 22

### Methyl 4-{6-[(2-chlorophenyl)amino]-1H-indazol-3-yl}benzoate Dihydrochloride

Prepared according to method 1. Starting from 3-iodo-6-nitro-indazole-1-carboxylic acid tert-butyl ester and 4-(methoxycarbonyl)phenylboronic acid to afford tert-butyl 3-[4-(methoxycarbonyl)phenyl]-6-nitro-1H-indazole-1-carboxylate. The nitro group was reduced and reacted with 1-bromo-2-chlorobenzene to yield the intermediate tert-butyl 6-[(2-chlorophenyl)amino]-3-[4-(methoxycarbonyl)phenyl]-1H-indazole-1-carboxylate, which gave after deprotection 38 mg of the title compound as an off-white solid. ¹H NMR (CD₃OD): δ 3.95 (s, 3H) 6.84 (s, 1H) 7.13 (m, 1H) 7.19 (m, 1H) 7.32 (m, 1H) 7.48 (m, 2H) 7.97 (m, 1H) 8.02 (m, 2H) 8.22 (m, 2H). MS C(SP) in I/z 378 (M+1).

### Example 23

### 4-{6-[(2-Chlorophenyl)amino]-H-indazol-3-yl}benzoic Acid Dihydrochloride

A solution of tert-butyl 6-[(2-chlorophenyl)amino]-3-[4-(methoxycarbonyl)phenyl]-1H-indazole-1-carboxylate, from example 22, (680 mg, 1.42 mmol) in THF (15 mL) and 6 M HCl (50 mL) was stirred for 48 h at 80° C. The solvents were evaporated in vacuo and the residue was treated with diethyl ether to precipitate 155 mg of the title compound. ¹H NMR (DMSO-D6): δ 7.01 (m, 2H) 7.04 (m, 1H) 7.27 (m, 1H) 7.39 (m, 1H) 7.48 (m, 1H) 7.97 (m, 1H) 8.04 (m, 21) 8.10 (m, 2H). MS (TSP) m/z 364 (M+1).

### Example 24

### Methyl 3-{6-[(2-chlorophenyl)amino]-1H-indazol-3-yl}benzoate Dihydrochloride

Prepared according to method 1. Starting from 3-iodo-6-nitro-indazole-1-carboxylic acid tert-butyl ester and 3-(methoxycarbonyl)phenylboronic acid to afford tert-butyl 3-[3-(methoxycarbonyl)phenyl]-6-nitro-1H-indazole-1-carboxylate. The nitro group was reduced and reacted with 1-bromo-2-chlorobenzene to yield the intermediate tert-butyl 6-[(2-chlorophenyl)amino]-3-[3-(methoxycarbonyl)phenyl]-1H-indazole-1-carboxylate, which gave after deprotection 14 mg of the title compound as an off-white solid. ¹H NMR (CD₃OD): δ 3.88 (s, 3H) 6.83 (m, 1H) 7.01 (m, 1H) 7.09 (m, 1H) 7.21 (m, 1H) 7.40 (m, 2H) 7.64 (m, 1H) 7.86 (m, 1H) 8.09 (m, 2H) 8.47 (s, 1H). MS (TSP) m/z 378 (M+1).

### Example 25

### 3-(6-[(2-Chlorophenyl)amino]-1H-indazol-3-yl}benzoic Acid Dihydrochloride

A solution of tert-butyl 6-[(2-chlorophenyl)amino]-3-[3-(methoxycarbonyl)phenyl]-1H-indazole-1-carboxylate, from example 24, (540 mg, 0.872 mmol) in THF (6 mL) and 6M HCl (10 mL) was stirred for 48 h at 80° C. The solvents were evaporated in vacuo and the residue was treated with diethyl ether to precipitate 160 mg of the title compound. ¹H NMR (DMSO-D6): δ 7.03 (m, 3H) 7.28 (m, 1H) 7.40 (m, 1H) 7.49 (m, 1H) 7.64 (m, 1H) 7.93 (m, 2H) 8.21 (m, 1H) 8.53 (s, 1H). MS (TSP) m/z 364 (M+1).

### Example 26

### N-(2-chlorophenyl)-3-{3-[(4-methylpiperazin-1-yl)carbonyl]phenyl}-1H-indazol-6-amine

3-{6-[(2-Chlorophenyl)amino]-1H-indazol-3-yl}benzoic acid dihydrochloride (Example 25, 44 mg, 0.1 mmol) was dissolved in a solution of N-methylpiperazine (100 mM, 1.2 mL, 0.12 mmol). N-cyclohexylcarbodiimide,N′-methyl polystyrene (1.15 mmol/g, 261 mg, 0.3 mmol), N,N-(diisopropyl)aminomethylpolystyrene (3.83 mmole/g, 261 mg, 0.3 mmol) and N-hydroxybenzotriazole (16 mg, 0.12 mmol) was added. The mixure was stirred under nitrogen for 16 h at room temperature until HPLC/MS shows that all carboxylic acid has been consumed. The mixture was filtered, evaporated under reduced pressure, and chromatographed on silica in dichloromethane/methanol 10:1. Appropriate fractions were combined and concentrated to dryness to yield 36 mg (81%) of the title compound. ¹H NMR (CDCl₃) δ 8.00 (s, 1H), 7.99 (d, J=8.9 Hz, 1H), 7.85 (d, J=8.9 Hz, 1H), 7.51 (t, J=7.86 Hz, 1H), 7.43-7.32 (m, 3H), 7.14 (t, J=7.68 Hz, 1H), 7.10 (s, 1H), 6.98 (dd, J=1.6/8.7 Hz, 1H), 6.85 (t, 7.68 Hz, 1H), 6.28 (s, 1H), 3.84 (bs, 2H), 3.50 (bs, 2H), 2.49 (br s, 2H), 2.33 (bs, 2H), 2.30 (s, 3H). ¹³C NMR (CDCl₃) δ 170.2, 162.6, 144.7, 143.0, 141.3, 139.8, 136.4, 134.1, 129.9, 129.0, 128.6, 127.5, 126.5, 125.9, 122.5, 121.8, 121.3, 116.9, 116.5, 98.0, 55.3, 54.7, 47.7, 46.0, 42.0.

HPLC-MS (Waters Exterra C8-column, 8.6 min gradient of 0-100% methanol containing 0.1% trifluoroacetic acid. UV-diode array detector, CLND and MSD-ESI detection) shows a single compound with m/z 446 (M+1). C25H24ClN50, MW=446.0.

### Example 27-41

Indazoles 27-41 were prepared essentially according to the procedure described for Example 26. Thus, a solution of 4-{6-[(2-Chlorophenyl)amino]-1H-indazol-3-yl}benzoic acid dihydrochloride (Example 23, 13.3 mg, 0.03 mmol) in 200 uL dimethylformamide is added to a polypropylene fritted vessel (Bohdan Miniblock system). Amine (100 mM in dimethylformamide (370 uL, 0.037 mmol) is added, followed by the polystyrene resins N-cyclohexylcarbodiimide, N′-methyl polystyrene (1.15 mmol/g, 54 mg, 0.062 mmol) and N,N-(diisopropyl)aminomethylpolystyrene (3.72 mmol/g, 25 mg, 0.093 mmol). 1 mL of dry chloroform is added, the tubes are flushed with argon, sealed and agitated on an orbital shaker for 16 h at 50° C. The solution is filtered and the resin mixture is washed with dichloromethane and methanol (1+1 mL). The combined filtrate is evaporated to dryness in a vacuum centrifuge, and chromatographed on a preparative HPLC/MS system (Waters 2767/2525) with a gradient of 30%-100% acetonitrile in 0.05 M aqueous ammonium acetate. Appropriate fractions were combined and concentrated to dryness to yield the title compounds.

HPLC-MS (Waters Exterra C8-column, 8.6 min gradient of 0-100% methanol containing 0.1% trifluoroacetic acid. UV-diode array detector, CLND and MSD-ESI detection) shows pure compounds with m/z according to the below table.

| Example | Name | MW | Found m/z | Amount (mg) |
|---|---|---|---|---|
| 27 | 4-{6-[(2-chlorophenyl)amino]-1H- | 503.0 | 503 | 0.1 |
| | indazol-3-yl}-N-[3-(4-methylpiperazin-1- | | | |
| | yl)propyl]benzamide | | | |
| 28 | 4-{6-[(2-chlorophenyl)amino]-1H- | 476.0 | 476 | 0.2 |
| | indazol-3-yl}-N-(2-morpholin-4- | | | |
| | ylethyl)benzamide | | | |
| 29 | 4-{6-[(2-chlorophenyl)amino]-1H- | 433.9 | 434 | 0.4 |
| | indazol-3-yl}-N-[2- | | | |
| | (dimethylamino)ethyl]benzamide | | | |
| 30 | 4-{6-[(2-chlorophenyl)amino]-1H- | 448.0 | 448 | 0.1 |
| | indazol-3-yl}-N-[3- | | | |
| | (dimethylamino)propyl]benzamide | | | |
| 31 | 4-{6-[(2-chlorophenyl)amino]-1H- | 419.9 | 420 | 0.1 |
| | indazol-3-yl}-N-[3-carbamoylmethyl- | | | |
| | benzamide | | | |
| 32 | N-(2-chlorophenyl)-3-[4-(thiomorpholin- | 449.0 | 449 | 2 |
| | 4-ylcarbonyl)phenyl]-1H-indazol-6- | | | |
| | amine | | | |
| 33 | methyl N-(4-{6-[(2-chlorophenyl)amino]- | 448.9 | 449 | 0.8 |
| | 1H-indazol-3-yl}benzoyl)-N- | | | |
| | methylglycinate | | | |
| 34 | 1-(4-{6-[(2-chlorophenyl)amino]-1H- | 432.9 | 433 | 1.5 |
| | indazol-3-yl}benzoyl)pyrrolidin-3-ol | | | |
| 35 | 4-{6-[(2-chlorophenyl)amino]-1H- | 440.9 | 441 | 0.2 |
| | indazol-3-yl}-N,N- | | | |
| | bis(cyanomethyl)benzamide | | | |
| 36 | N-(2-chlorophenyl)-3-(4-{[3- | 460.0 | 460 | 1.8 |
| | (dimethylamino)pyrrolidin-1- | | | |
| | yl]carbonyl}phenyl)-1H-indazol-6-amine | | | |
| 37 | 4-{6-[(2-chlorophenyl)amino]-1H- | 462.0 | 462 | 0.1 |
| | indazol-3-yl}-N-[2- | | | |
| | (dimethylamino)ethyl]-N-ethylbenzamide | | | |
| 38 | 1-(4-{6-[(2-chlorophenyl)amino]-1H- | 474.0 | 474 | 1 |
| | indazol-3-yl}benzoyl)piperidine-4- | | | |
| | carboxamide | | | |
| 39 | 4-{6-[(2-chlorophenyl)amino]-1H- | 420.9 | 421 | 1 |
| | indazol-3-yl}-N-(2-hydroxyethyl)-N- | | | |
| | methylbenzamide | | | |
| 40 | 1-(4-{6-[(2-chlorophenyl)amino]-1H- | 446.9 | 447 | 1.7 |
| | indazol-3-yl}benzoyl)piperidin-4-ol | | | |
| 41 | N-(2-chlorophenyl)-3-[4-(morpholin-4- | 432.9 | 433 | 3.1 |
| | ylcarbonyl)phenyl]-1H-indazol-6-amine | | | |

### Example 42-59

The compound in example 25, 3-{6-[(2-chlorophenyl)amino]-1H-indazol-3-yl}benzoic acid dihydrochloride, was chromatographed on reversed phase silica to obtain the free carboxylic acid in pure form. This carboxylic acid (10.9 mg, 0.03 mmol) in 1200 uL dimethylformamide is added to a round bottom glass tube (Bohdan Miniblock XT system). Primary amine (100 mM in dimethylformamide (540 uL, 0.054 mmol) is added, followed by diisopropylcarbodiimide (8.4 uL, 0.054 mmol) and N-hydroxybenzotriazole (4.9 mg, 0.036 mmol). The mixture is stirred 48 h at room temperature under argon, evaporated to dryness in a vacuum centrifuge, and chromatographed on a preparative aqueous ammonium HPLC/MS system (Waters 2767/2525) with a gradient of 20%-100% acetonitrile in 0.1 M ammonium acetate. Appropriate fractions were combined and concentrated to dryness to yield the title compounds.

HPLC-MS (Waters Exterra C8-column, 7.1 min gradient of 0-100% acetonitrile in 10 mM ammonium acetate. UV-diode array detector and MSD-ESI detection shows pure compounds with m/z according to the below table.

| Example | Name | MW | Found m/z | Amount (mg) |
|---|---|---|---|---|
| 42 | 3-{6-[(2-chlorophenyl)amino]-1H- | 478.0 | 478 | 10.7 |
| | indazol-3-yl}-N-{3-[(2- | | | |
| | hydroxyethyl)(methyl)amino]propyl}benzamide | | | |
| 43 | 3-{6-[(2-chlorophenyl)amino]-1H- | 490.0 | 490 | 11.4 |
| | indazol-3-yl}-N-(3-morpholin-4- | | | |
| | ylpropyl)benzamide | | | |
| 44 | 3-{6-[(2-chlorophenyl)amino]-1H- | 476.0 | 476 | 4.5 |
| | indazol-3-yl}-N-[2-(diethylamino)-1- | | | |
| | methylethyl]benzamide | | | |
| 45 | 3-{6-[(2-chlorophenyl)amino]-1H- | 450.9 | 451 | 1.0 |
| | indazol-3-yl}-N-[2-hydroxy-1- | | | |
| | (hydroxymethyl)-1- | | | |
| | methylethyl]benzamide | | | |
| 46 | 3-{6-[(2-chlorophenyl)amino]-1H- | 476.0 | 476 | 8.7 |
| | indazol-3-yl}-N-(2-morpholin-4- | | | |
| | ylethyl)benzamide | | | |
| 47 | ethyl 4-[(3-{6-[(2-chlorophenyl)amino]- | 518.0 | 518 | 7.4 |
| | 1H-indazol-3- | | | |
| | yl}benzoyl)amino]piperidine-1- | | | |
| | carboxylate | | | |
| 48 | 3-{6-[(2-chlorophenyl)amino]-1H- | 474.0 | 474 | 49 |
| | indazol-3-yl}-N-(2-piperidin-1- | | | |
| | ylethyl)benzamide | | | |
| 49 | 3-{6-[(2-chlorophenyl)amino]-1H- | 433.9 | 434 | 15.2 |
| | indazol-3-yl}-N-[2- | | | |
| | (dimethylamino)ethyl]benzamide | | | |
| 50 | 3-{6-[(2-chlorophenyl)amino]-1H- | 448.0 | 448 | 8.4 |
| | indazol-3-yl}-N-[3- | | | |
| | (dimethylamino)propyl]benzamide | | | |
| 51 | 3-{6-[(2-chlorophenyl)amino]-1H- | 434.9 | 435 | 6.1 |
| | indazol-3-yl}-N-(2- | | | |
| | ethoxyethyl)benzamide | | | |
| 52 | 3-{6-[(2-chlorophenyl)amino]-1H- | 406.9 | 407 | 6.2 |
| | indazol-3-yl}-N-(2- | | | |
| | hydroxyethyl)benzamide | | | |
| 53 | N-[2-(acetylamino)ethyl]-3-{6-[(2- | 447.9 | 448 | 7.1 |
| | chlorophenyl)amino]-1H-indazol-3- | | | |
| | yl}benzamide | | | |
| 54 | 3-{6-[(2-chlorophenyl)amino]-1H- | 419.9 | 420 | 3.8 |
| | indazol-3-yl}-N-carbamoylmethyl- | | | |
| | benzamide | | | |
| 55 | 3-{6-[(2-chlorophenyl)amino]-1H- | 474.0 | 474 | 1.2 |
| | indazol-3-yl}-N-(1-ethylpiperidin-3- | | | |
| | yl)benzamide | | | |
| 56 | 3-{6-[(2-chlorophenyl)amino]-1H- | 474.0 | 474 | 11.2 |
| | indazol-3-yl}-N-(3-pyrrolidin-1- | | | |
| | ylpropyl)benzamide | | | |
| 57 | 3-{6-[(2-chlorophenyl)amino]-1H- | 503.0 | 503 | 1 |
| | indazol-3-yl}-N-[3-(4-methylpiperazin- | | | |
| | 1-yl)propyl]benzamide | | | |
| 58 | 3-{6-[(2-chlorophenyl)amino]-1H- | 474.0 | 474 | 2.9 |
| | indazol-3-yl}-N-[(1-ethylpyrrolidin-2- | | | |
| | yl)methyl]benzamide | | | |
| 59 | 3-{6-[(2-chlorophenyl)amino]-1H- | 446.9 | 447 | 3 |
| | indazol-3-yl}-N-(tetrahydrofuran-2- | | | |
| | ylmethyl)benzamide | | | |

### Example 60

### (2-Chloro-phenyl)-(5-methyl-3-phenyl-1H-indazol-6-yl)-amine Hydrochloride

### (i) 2,4-Dimethyl-5-nitro-phenylamine

To a solution of 2,4-dimethylaniline (1.50 g, 12.4 mmol) in diethyl ether (40 mL) was added conc. sulfuric acid to precipitate the aniline sulfate, which was filtered off and dried. A solution of the aniline sulfate in conc. sulfuric acid (5 mL) was then added to an ice-cold solution of potassium nitrate in conc. sulfuric acid (16 mL). The reaction mixture was allowed to reach ambient temperature and then stirred for 18 h. The mixture was then poured over ice (200 mL) and neutralized with conc. ammonium hydroxide (65 mL). The yellow precipitate formed was filtered off, washed with water and dried to afford 1.83 g (89%) of the title compound as an orange solid. LC-MS (API-ES) m/z 167.1 (M+1).

### (ii) 5-Methyl-6-nitro-1H-indazole

A solution of 2,4-dimethyl-5-nitro-phenylamine (1.83 g, 11.0 mmol) in 175 mL of acetic acid was treated with a solution of sodium nitrite (0.760 g, 11.0 mmol) in 3 mL of water and allowed to react for 3 days at ambient temperature. The acetic acid was removed in vacuo, leaving an oily residue to which 45 mL of water was added. The solid that precipitated was filtered off and dried to afford 1.75 g (90%) of the product as a red-brown solid. ¹H NMR (CDCl₃ Ref 7.26 ppm): δ 2.66 (3H, s), 5.47 (1H, br s), 7.72 (1H, s), 8.14 (1H, s), 8.20 (1H, s). LC-MS (API-ES) m/z 178.1 (M+1).

### (iii) 3-Iodo-5-methyl-6-nitro-1H-indazole

Iodine (4.44 g, 17.5 mmol) and potassium hydroxide pellets (1.85 g, 32.9 mmol) were successively added into a DMF solution of 5-methyl-6-nitro-1H-indazole (1.55 g, 8.73 mmol) under stirring at ambient temperature. After 2.5 h, the reaction mixture was poured into 10% aqueous NaHSO₃ (75 mL) and extracted with dichloromethane (3×). The combined organic phases were washed with (water and brine), dried (MgSO₄), filtered and the solvent evaporated. A small amount of dichloromethane was added and the solid was filtered, washed with dichloromethane and dried to give 0.810 g of the title compound as a yellow solid. The filtrate was concentrated in vacuo and the residue purified by flash chromatography (n-heptane/ethyl acetate 70/30) to yield an additional 0.534 g (50% in total) of the product as a yellow solid. ¹H NMR (CD₃OD): δ 2.61 (3H, s), 7.38 (1H, s), 8.11 (1H, s). LC-MS (API-ES) m/z 304 (M+1).

### (iv) 3-Iodo-5-methyl-6-nitro-indazole-1-carboxylic Acid Tert-Butyl Ester

To a solution of 3-iodo-5-methyl-6-nitro-1H-indazole (1.34 g, 4.41 mmol) in acetonitrile (40 mL) and methanol (20 mL) were added DMAP (0.054 g, 0.442 mmol), triethylamine (0.7 mL, 4.85 mmol) and di-tert-butyldicarbonate (1.06 g, 4.85 mmol) and the reaction was stirred at ambient temperature for 5 h. Water and dichloromethane were added, and the layers separated. The aqueous phase was extracted with dichloromethane (3×). The combined organic phases were washed (water, NaHCO₃ (sat. aq.), and brine), dried (MgSO₄), filtered and the solvent removed in vacuo to afford 1.68 g (94%) of the title compound as a pale yellow solid. ¹H NMR (CDCl₃ Ref 7.26 ppm): δ 1.73 (9H, s), 2.68 (3H, s), 7.44 (1H, s), 8.70 (1 h. s). LC-MS (API-ES) m/z 404 (M+1).

### (v) 5-Methyl-6-nitro-3-phenyl-indazole-1-carboxylic Acid Tert-Butyl Ester

To a mixture of 3-iodo-5-methyl-6-nitro-indazole-1-carboxylic Acid Tert-Butyl Ester (1.67 g, 4.15 mmol) and PdCl₂(dppf) (0.3 g, 0.4 mmol) in toluene/ethanol 10/1 (100 mL), Na₂CO₃ (sat. aq.) (35 mL) was added followed by the addition of phenylboronic acid (0.557 g, 4.57 mmol). Water and dichloromethane were added and the layers separated.

The aqueous phase was extracted with dichloromethane (3×). The combined organic phases were washed with water and brine, dried (MgSO₄), filtered and the solvent evaporated. The crude material was purified by flash chromatography (n-heptane/dichloromethane 50/50) to give 1.44 g (98%) of the title compound as a pale yellow solid.

¹H NMR (CDCl₃ Ref 7.26 ppm): δ 1.76 (9H, s), 2.69 (3H, s), 7.55 (3H, m), 7.90 (1H, s), 7.96 (2H, m), 8.79 (1H, s). ¹³C NMR (CDCl₃): δ 20.4, 28.1, 86.0, 111.7, 124.4, 126.3, 128.1, 128.2, 129.0, 129.8, 131.0, 138.7, 148.6, 149.2, 149.9. LC-MS (API-ES) m/z 354 (M+1).

### (vi) 6-Amino-5-methyl-3-phenyl-indazole-1-carboxylic Acid Tert-Butyl Ester

A mixture of 5-methyl-6-nitro-3-phenyl-indazole-1-carboxylic Acid Tert-Butyl Ester (1.44 g, 4.08 mmol) and PtO₂ (0.093 g, 0.41 mmol) in ethyl acetate/ethanol/tetrahydrofuran 1/1/1 (24 mL) was stirred under an atmosphere of hydrogen at ambient temperature for 4 h. The reaction mixture was filtered through a plug of celite and the solvents were evaporated to give 1.31 g (100%) of the title compound as a yellow solid. ¹H NMR (CDCl₃ Ref 7.26 ppm): δ 1.73 (9H, s), 2.30 (3H, s), 7.47 (4H, m), 7.61 (1H, s), 7.96 (2H, m). LC-MS (API-ES) m/z 324 (M+1).

### (vii) 6-(2-Chloro-phenylamino)-5-methyl-3-phenyl-indazole-1-carboxylic Acid Tert-Butyl Ester

Pd(OAc)₂ (92.0 mg, 0.406 mmol) and (S)-BINAP (380 mg, 0.609 mmol) were premixed in dry tetrahydrofuran (20 mL) for 5 min. under an atmosphere of nitrogen. 1-Bromo-2-chlorobenzene (620 μL, 5.28 mmol) and 6-amino-5-methyl-3-phenyl-indazole-1-carboxylic acid tert-butyl ester (1.31 g, 4.06 mmol) were added followed by cesium carbonate (1.85 g, 5.69 mmol). The reaction was stirred at 60° C. for 18 h under an atmosphere of nitrogen. Additional Pd(OAc)₂ (92.1 mg, 0.406 mmol), (S)-BINAP (380 mg, 0.609 mmol) and 1-bromo-2-chlorobenzene (620 μL, 5.28 mmol) were added and the reaction was stirred at 60 IC for 7 h under an atmosphere of nitrogen. Due to the low reactivity, additional Pd(OAc)₂ (92.0 mg, 0.406 mmol) and (S)-BINAP (380 mg, 0.609 mmol) were added and the reaction was stirred for another 20 h at 60° C. under an atmosphere of nitrogen. Water and dichloromethane were added and the layers separated.

The aqueous phase was extracted with dichloromethane (3×). The combined organic phases were washed with water and brine, dried (MgSO₄), filtered and the solvent evaporated. The crude material was purified by flash chromatography (n-heptane/ethyl acetate 80/20) to give 637 mg (36%) of the title compound as a yellow solid. ¹H NMR (CDCl₃): δ 1.65 (9H, s), 2.44 (3H, s), 6.12 (1H, br s), 6.92 (1H, m), 7.22 (1H, m), 7.47 (5H, m), 7.79 (1H, s), 8.00 (3H, m). LC-MS (API-ES) in/z 434 and 436 (M+1).

### (viii) (2-Chloro-phenyl)-(5-methyl-3-phenyl-1H-indazol-6-yl)-amine Hydrochloride

To a solution of 6-(2-chloro-phenylamino)-5-methyl-3-phenyl-indazole-1-carboxylic acid tert-butyl ester (77.0 mg, 0.177 mmol) in methanol (2 mL) was added 4 M HCl in diethylether (2 mL) and the reaction was stirred at ambient temperature for 24 h. The solvent was evaporated and the solid residue was recrystallized to yield 48.1 mg of the title compound as a pale red-brown solid. ¹H NMR (CD₃OD): δ 2.46 (3H, s), 7.01 (1H, s), 7.10 (1H, dt), 7.31 (1H, dt), 7.47 (2H, m), 7.60 (3H, m), 7.79 (1H, s), 7.85 (2H, m).

LC-MS (API-ES) m/z 334 and 336 (M+1).

### Example 61

### N-(2-morpholin-4-ylethyl)-6-nitro-3-phenyl-1H-indazol-5-amine Hydrochloride

### (i) 5-bromo-6-nitro-1-{[2-trimethylsilyl)ethoxy]methyl}-1H-indazole

5-bromo-6-nitro-1H-indazol (ref. Foster R. H., Leonard N. J., J. Org. Chem., 1979, 44 4609) (668 mg, 2.76 mmol) was dissolved THF (10 mL) and cooled to 0° C. Sodium tert-butoxide (320 mg, 3.31 mmol) was added and the mixture was stirred at 0° C. for 0.5 h. 2-(trimethylsilyl)ethoxymethyl chloride (587 μL, 3.31 mmol) was then added and the stirring continued at 0° C. for 1 h. The solution was diluted with ethyl acetate (40 mL), washed with water (40 mL) and brine (40 mL). The organic layer was dried (Na₂SO₄), filtered, concentrated and purified by silica gel chromatography (n-heptane/ethyl acetate 4:1) to give 544 mg (53%) of the title compound. ¹H NMR (CDCl₃): δ −0.07 (9H, s), 0.88 (2H, t), 3.52 (2H, t), 5.75 (2H, s), 8.05 (1H, s), 8.10 (2H, s). ¹³C NMR (CDCl₃): δ 0.0, 19.2, 68.5, 79.9, 106.8, 109.3, 128.5, 134.8, 138.5, 141,9. MS(ECI) m/z 372 and 374 (M+1).

### (ii) N-(2-morpholin-4-ylethyl)-6-nitro-1-{[2-trimethylsilyl)ethoxy]methyl}-1H-indazol-5-amine

Pd(OAc)₂ (9.3 mg, 0.043 mmol) and (±)-BINAP (38 mg, 0.064 mmol) were premixed in dry toluene (1 mL) for 5 min, under an atmosphere of nitrogen. 2-(aminoethyl)morpholine (212 μL, 2.10 mmol) and 5-bromo-6-nitro-1-{[2-trimethylsilyl)ethoxy]methyl}-1H-indazole (77 mg, 0.21 mmol) were added followed by cesium carbonate (94 mg, 0.29 mmol) addition. The reaction was stirred at 80° C. for 5 h under an atmosphere of nitrogen, then filtered through celite and purified by silica gel chromatography (n-heptane/ethyl acetate+1% TEA, 2:1→1:3) to give 61 mg (70%) of the title compound. ¹H NMR (CDCl₃): δ −0.07 (9H, s), 0.87 (2H, t), 2.53 (4H, t), 2.76 (2H, tr, 3.32 (2H, t), 3.52 (2H, t), 3.76 (4H, t), 5.68 (2H, s), 6.96 (1H, s), 7.74 (1H, s), 7.78 (1H, s), 8.48 (1H, s). ¹³C NMR (CDCl₃): δ −1.50, 17.7, 40.1, 53.2, 56.2, 66.6, 67.0, 77.9, 101.6, 108.4, 130.4, 131.5, 132.6, 135.0, 139.8.

MS(ECI) m/z 422 (M+1).

### (iii) N-(2-morpholin-4-ylethyl)-6-nitro-1H-indazole-5-amine

Tetrabutylammonium fluoride (1.0 M in THF, 5 mL, 5.0 mmol) and 1,2-diaminoethene (166 μL, 2.5 mmol) were added to N-(2-morpholin-4-ylethyl)-6-nitro-1-{[2-trimethylsilyl)ethoxy] methyl}-1H-indazole-5-amine (105 mg, 0.25 mmol). The reaction mixture was heated overnight at 70° C. After cooling to ambient temperature, the reaction mixture was diluted with ethyl acetate (25 mL) and washed with saturated aqueous NaCO₃ (25 mL). The organic layer was dried (Na₂SO₄), filtered, concentrated and purified by silica gel chromatography (n-heptane/ethyl acetate+1% TEA, 1:1→1:15) to give 64 mg (88%) of the title compound. ¹H NMR (CDCl₃): δ 2.54 (4H, tr), 2.76 (2H, tr), 3.33 (2h, q), 3.76 (4H, tr), 6.98 (1H, s), 7.69 (1H, s), 7.96 (1H, s), 8.41 (1H, s), 10.22 (1H, s). ¹³C NMR (CDCl₃): 640.1, 53.2, 56.2, 67.0, 101.3, 108.4, 128.9, 131.8, 133.8, 135.3, 139.7.

MS(ECI) m/z 292 (M+1).

### (iv) 3-iodo-N-(2-morpholin-4-ylethyl)-6-nitro-1H-indazol-5-amine

Iodine (112 mg, 0.44 mmol) and potassium hydroxide (46 mg, 0.82 mmol) were successively added to a solution of N-(2-morpholin-4-ylethyl)-6-nitro-1H-indazole-5-amine (64 mg, 0,22 mmol) in DMF (10 mL) at ambient temperature. The reaction mixture was stirred for 2 h and then diluted with ethyl acetate (30 mL) and washed with 10% aqueous NaHSO₃ (25 mL) and brine (25 mL). The organic layer was dried (Na₂SO₄), filtered, concentrated and purified by silica gel chromatography (toluene/ethyl acetate, 1:1) to give 66 mg (72%) of the title compound. ¹H NMR (CDCl₃): δ 2.55 (4H, t), 2.78 (2H, t), 3.37 (2H, t), 3.77 (4H, t), 6.68 (1H, s), 7.77 (1H, s), 8.42 (1H, s), 10.58 (1H, s).

MS(ECI) m/z 418 (M+1).

### (v) tert-butyl 3-iodo-5-[(2-morpholin-4-ylethyl)amino]-6-nitro-1H-indazole-1-carboxylate

Di-tert-butyl dicarbonate (42 mg, 0.19 mmol) and DMAP (2 mg, 0.016 mmol) were added to a solution of 3-iodo-N-(2-morpholin-4-ylethyl)-6-nitro-1H-indazole-5-amine (66 mg, 0.16 mmol) in acetonitrile (10 mL) at ambient temperature. The reaction mixture was stirred for 0.5 h, then concentrated and purified by silica gel chromatography (toluene/ethyl acetate, 3:1→1:1) to give 79 mg (97%) of the title compound. ¹H NMR (CDCl₃): δ 1.71 (9H, s), 2.54 (4H, s), 2.77 (2H, t), 3.37 (2H, t), 3.76 (4H, t), 6.66 (1H, s), 8.02 (1H, s), 8.93 (1H, s). ¹³C NMR (CDCl₃): δ 28.1, 39.8, 53.2, 55.9, 67.0, 86.0, 101.0, 103.3, 113.4, 129.3, 135.2, 135.8, 141.6, 147.6, 171.1.

### (vi) tert-butyl 3-iodo-5-[(2-morpholin-4-ylethyl)amino]-6-nitro-3-phenyl-1H-indazole-1-carboxylate

To a mixture of tert-butyl 3-iodo-5-[(2-morpholin-4-ylethyl)amino]-6-nitro-1H-indazole-1-carboxylate (79 mg, 0.15 mmol) and PdCl₂(dppf) (11.2 mg, 0.015 mmol) in toluene/ethanol/water 10/1/1.5 (12.5 mL), Na₂CO₃ (49 mg, 0.46 mmol) was added followed by the addition of phenylboronic acid (26 mg, 0.21 mmol). The reaction was stirred at 80° C. for 5 h under an atmosphere of nitrogen. Water (10 mL) and ethyl acetate (10 mL) were added and the layers separated. The aqueous phase was extracted with ethyl acetate (3×). The combined organic phases were washed with water and brine, dried (Na₂SO₄), filtered, concentrated and purified by silica gel chromatography (n-heptane/ethyl acetate, 3:1→1:1) to give 71 mg (99%) of the title compound. ¹H NMR (CDCl₃): δ 1.74 (9H, s), 2.53 (4H, s), 2.76 (2H, t), 3.35 (2H, q), 3.76 (4H, t), 7.16 (1H, s), 7.51 (3H, m), 7.91 (2H, s), 8.01 (1H, s), 9.00 (1H, s). ¹³C NMR (CDCl₃): δ 28.1, 39.8, 53.2, 56.0, 67.0, 85.4, 102.8, 113.4, 128.0, 129.0, 129.7, 129,8, 131.3, 134.8, 141.6, 148.7, 171.1.

MS(ECI) m/z 468 (M+1).

### (vii) N-(2-morpholin-4-ylethyl)-6-nitro-3-phenyl-1H-indazol-5-amine Hydrochloride

To a solution of tert-butyl 3-iodo-5-[(2-morpholin-4-ylethyl)amino]-6-nitro-3-phenyl-1H-indazole-1-carboxylate (71 mg, 0.15 mmol) in methanol/THF (1.5:0.5, 2 mL) was added 4 M HCl in diethylether (1 mL) and the reaction was stirred at ambient temperature for 24 h. The solvent was evaporated, the crude mixture was purified by precipitation from a methanol/THF/diethyl ether (1:0.5:3) solution to give 41 mg (57%) of the title compound. ¹H NMR (CDCl₃): δ 3.34 (2H, m), 3.56 (2H, m), 3.61 (2H, m), 3.80 (2H, m), 3.88 (2H, m), 4.04 (2H, m), 7.38 (1H, s), 7.43 (1H, m), 7.54 (2H, m), 7.94 (2H, m), 8.48 (1H, s).

MS (TSP) m/z 368 (M+1).

### Example 62

### (2-Fluoro-phenyl)-(3-phenyl-1H-indazol-6-yl)-amine Hydrochloride

### (i) 6-(2-Fluoro-phenylamino)-3-phenyl-indazole-1-carboxylic Acid Tert-Butyl Ester

Pd(OAc)₂ (22 mg, 0.098 mmol) and (S)-BINAP (92 mg, 0.148 mmol) were premixed in dry tetrahydrofuran (4 mL) for 5 min. under an atmosphere of nitrogen. 1-Bromo-2-fluorobenzene (100 μL, 0.889 mmol) and 6-amino-3-phenyl-indazole-1-carboxylic acid tert-butyl ester (250 mg, 0.808 mmol) were added followed by cesium carbonate (369 mg, 1.13 mmol). The reaction was stirred at 60° C. for 7.5 h under an atmosphere of nitrogen. Additional Pd(OAc)₂ (22 mg, 0.098 mmol), (S)-BINAP (92 mg, 0.148 mmol) and 1-bromo-2-fluorobenzene (100 μL, 0.889 mmol) were added and the reaction was stirred at 60° C. for 18 h under an atmosphere of nitrogen. Some starting material left according to TLC, so additional Pd(OAc)₂ (22 mg, 0.098 mmol) and (S)-BINAP (92 mg, 0.148 mmol) were added and the reaction was stirred for another 8 h at 60° C. under an atmosphere of nitrogen. Water and dichloromethane were added and the layers separated. The aqueous phase was extracted with dichloromethane (3×). The combined organic phases were washed with water and brine, dried (MgSO₄), filtered and the solvent evaporated. The crude material was purified by flash chromatography (petroleum ether/EtOAc 90/10) to afford 64 mg (20%) of the title compound as a yellow solid. ¹H NMR (CDCl₃ Ref 7.26 ppm): δ 1.68 (9H, s), 6.97 (1H, m), 7.06 (1H, dd), 7.13 (2H, m), 7.49 (4H, m), 7.85 (1H, d), 7.88 (1H, s), 7.99 (2H, m). LC-MS (API-ES) m/z 404.1 (M+1).

### (ii) (2-Fluoro-phenyl)-(3-phenyl-1H-indazol-6-yl)-amine Hydrochloride

To a solution of 6-(2-fluoro-phenylamino)-3-phenyl-indazole-1-carboxylic acid tert-butyl ester (60.0 mg, 0.149 mmol) in methanol (2 mL) was added 4 M hydrochloric acid in diethyl ether (1 mL) and the reaction stirred for 65 h at ambient temperature. Additional 4 M hydrochloric acid in diethyl ether (1 mL) was added and the reaction stirred for 24 h at ambient temperature. The solvent was evaporated and diethyl ether was added to the solid, which was filtered off, washed with diethyl ether and dried to afford 40.0 mg of the title compound as a pale brown solid. ¹H NMR (CD₃OD): δ 6.87 (1H, s), 7.14 (4H, m), 7.41 (1H, m), 7.60 (3H, m), 7.84 (3H, m). ¹³C NMR (CD₃OD): δ 112.9, 116.7, 116.9, 119.3, 123.2, 124.1, 125.0(6), 125.1(O), 125.6, 127.0, 128.6, 128.7, 128.8, 130.1, 131.7, 142.2, 143.5, 149.5, 154.9, 157.3. LC-MS (API-ES) m/z 304.1 (M+1).

### Example 63

### 3-(4-Methanesulfonyl-phenyl)-6-nitro-1H-indazole Hydrochloride

Prepared according to method 1a. Starting from 3-iodo-6-nitro-indazole-1-carboxylic acid tert-butyl ester and 4-(methanesulfonyl)phenylboronic acid to afford 3-(4-methanesulfonyl-phenyl)-6-nitro-indazole-1-carboxylic Acid Tert-Butyl Ester, which was deprotected to yield 54.0 mg of the title compound as a pale yellow solid. ¹H NMR (DMSO): δ 3.30 (3H, s), 8.07 (1H, dd), 8.10 (2H, d), 8.31 (2H, d), 8.40 (1H, d), 8.55 (1H, d), 14.24(1H, br s). ¹³C NMR (DMSO): δ 43.6, 107.8, 116.0, 121.9, 123.1, 127.5, 127.8, 137.3, 140.1, 140.4, 142.3, 146.0. LC-MS (API-ES) ir/z 318 (M+1).

### Example 64

### 3-Furan-3-yl-6-nitro-1H-indazole Hydrochloride

Prepared according to method 1a. Starting from 3-iodo-6-nitro-indazole-1-carboxylic acid tert-butyl ester and furan-3-boronic acid to afford 3-furan-3-yl-6-nitro-indazole-1-carboxylic acid tert-butyl ester, which was deprotected to give 53 mg of the title compound as a yellow solid. ¹H NMR (DMSO): δ 7.08 (1H, d), 7.87 (1H, s), 7.97 (1H, dd), 8.29 (1H, d), 8.47 (1H, d), 8.56 (1H, s). ¹³C NMR (DMSO): δ 107.3, 108.9, 114.9, 118.3, 121.9, 122.9, 137.7, 139.9, 140.6, 144.2, 146.0. LC-MS (API-ES) m/z 230 (M+1).

Biological Evaluation

The compounds of this invention may be assayed for their activity according to the following procedure:

A scintillation proximity assay (SPA) based on the inhibition of JNK3 catalyzed transfer of the γ-phosphate group of [γ-³³P] ATP to biotinylated ATF2, has been set up to identify inhibitory compounds. The resulting ³³P-labeled biotinylated ATF2 is trapped on SPA beads surface coated with streptavidin.

The assay is performed in 96-well plates. Test compounds made up at 10 mM in DMSO and 1:3 serial dilutions are made in 100% DMSO. These serial dilutions are then diluted 1:10 in assay buffer (50 mM MOPS pH 7.2, 150 mM, NaCl, 0.1 mM EGTA, 1 mM DTT, 6.25 mM β-glycerolphosphate) and 10 μl are transferred to assay plates (results in 2% DMSO final concentration in assay). To each well with test compound a 2.4 μl JNK3/ATP enzyme solution (1.18 U/ml JNK3, 20 μM ATP, 2 mM Mg(Ac)₂, 0.01% Brij-35 in assay buffer) was added. The mixture was pre-incubated for 10 minutes at ambient temperature. After this, 3.6 μl of a [γ-³³P] ATP-solution (0.20 μCi/μl [γ-³³P]ATP, 66.6 mM Mg(Ac)₂, 1 mM DTT, 50 mM MOPS pH 7.2, 150 mM NaCl, 0.1 mM EGTA) was added to each well followed by 10 μl of a ATF2 solution (60 μg/ml biotinylated ATF2 in assay buffer) to start the reaction. The reaction was allowed to proceed for 10 minutes at ambient temperature. After this, the reaction was terminated by the addition of 200 μl per well of stop buffer/bead mix (0.4 mg/ml streptavidin coated SPA-beads in 50 mM EDTA, pH 7.6). Plates were sealed with a plastic cover and centrifuged (2000 rpm, 5 minutes) to settle the beads followed by counting in a Wallac 1450 microbeta™.

The IC₅₀ values were calculated as the concentration of test compound at which the ATF2 phosphorylation is reduced to 50% of the control value.

Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 300 nM.

List of Abbreviations

| | |
|---|---|
| SPA | scintillation proximity assay |
| ATP | adenosine triphosphate |
| ATF | Activating transcription factor |
| MOPS | 3-[N-Morpholino]-propanesulfonic acid |
| EGTA | Ethylene glycol-bis(β-aminoethylether)- |
| | N,N,N′,N′-tetraacetic acid |
| DTT | dithiothreitol |
| JNK | Jun N-terminal kinases |
| MAP | mitogen-activated protein |

1. A compound of Formula I: 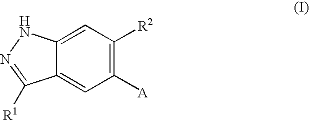
wherein:
R¹ is aryl or heteroaryl each of which is optionally substituted with one or more of the following R³, —OR³, —OCOR³, —COOR³, —COR³, —CONR³R⁴, —NHCOR³, —NR³R⁴, —NHSO₂R³, —SO₂R³, —SO₂NR³R⁴, —SR³, CN, halogeno or NO₂;
R² is NO₂, NH₂, —NR⁵R⁶ or —NR⁶R⁷;
R³ and R⁴ are each independently hydrogen, C₁₋₆alkyl, C₂₋₆alkenyl, (C₃₋₈cycloalkyl)C₀₋₆alkyl, C₁₋₆fluoroalkyl, heterocycleC₀₋₆alkyl, heteroarylC₀₋₆alkyl; and said C₁₋₆alkyl, C₂₋₆alkenyl, (C₃₋₈cycloalkyl)C₀₋₆alkyl, C₁₋₆fluoroalkyl, heterocycleC₀₋₆alkyl, heteroarylC₀₋₆alkyl may be substituted with one or more B;
or R³ and R⁴ form together a 5-, 6- or 7-membered heterocyclic ring containing 1 to 4 heteroatoms independently selected from N, O and S, and said ring may be substituted with one or more B;
B is R¹⁰, —COOR¹⁰, —COR¹⁰, —NHCOR¹⁰, —NR¹⁰R¹¹, —CONR¹⁰R¹, —OR¹⁰, —SO₂NR¹⁰R¹¹, CN, halogeno or oxo;
R⁵ is phenyl or heteroaryl each of which is optionally substituted with one or more of R¹⁰, —OR¹⁰, —OCOR¹⁰, —COOR¹⁰, —CONR¹⁰R¹¹, —NHCOR¹⁰, —NR¹⁰R¹¹, —NHSO₂R¹⁰, —SO₂R¹⁰, —SO₂NR¹⁰R¹¹, —SR¹⁰, CN, halogeno, or NO₂;
R⁶ is hydrogen, C₁₋₆alkyl, heterocycleC₀₋₆alkyl, or hydroxyC₁₋₆alkyl;
R⁷ is C₁₋₆alkyl, (C₃₋₈cycloalkyl)C₀₋₆alkyl, C₅₋₈cycloalkenylC₀₋₆alkyl, or R⁵C₁₋₆alkyl;
A is hydrogen, R⁸, —OR⁸, —OCOR⁸, —COOR⁸, —CONR⁸R⁹, —NHCOR⁸, —NR⁸R⁹, —NHSO₂R⁸, —SO₂R⁸, —SO₂NR⁸R⁹, —SR⁸, CN, halogeno, heterocycleC₀₋₆alkyl, or heteroarylC₀₋₆alkyl;
R⁸ and R⁹ each independently are hydrogen, C₁₋₆alkyl, C₂₋₆alkenyl, C₂₋₆alkynyl heterocycleC₀₋₆alkyl-, heteroarylC₀₋₆alkyl; and said C₁₋₆alkyl, C₂₋₆alkenyl, C₂ alkynyl heterocycleC₀₋₆alkyl, or heteroarylC₀₋₆alkyl may be substituted with one or more B;
or R⁸ and R⁹ form together a 5-, 6- or 7-membered heterocyclic ring containing 1 to 4 heteroatoms independently selected from N, O and S, and said ring may be substituted with one or more B;
R¹⁰ and R¹¹ each independently are hydrogen, C₁₋₆alkyl, C₁₋₆fluoroalkyl or hydroxyC₁₋₆alkyl, or;
R¹⁰ and R¹¹ form together a 5-, 6- or 7-membered heterocyclic ring containing 1 to 4 heteroatoms independently selected from N, O and S, and said ring may be substituted with one or more B;
with the proviso that said compound is not 6-amino-3-(4-fluorophenyl)-indazole, 6-amino-3-phenyl-indazole, 6-nitro-3-phenyl-indazole, 6-nitro-3-(4-nitrophenyl)-indazole and that said compounds has not a quinazoline in R⁵ position;
as a free base or a salt thereof.

2. A compound according to claim 1 as a pharmaceutically acceptable salt thereof.

3. A compound according to claim 1 or 2, wherein R¹ is aryl or heteroaryl each of which is optionally substituted with one or more of the following: —COOR³, —CONR³R⁴, —NHCOR³, or —NR³R⁴; R² is NO₂, NH₂, —NR⁵R⁶ or —NR⁶R⁷; R³ and R⁴ are each independently hydrogen or C₁₋₆alkyl or heterocycleC₀₋₆alkyl, and said C₁₋₆alkyl may be substituted with one or more B; or R³ and R⁴ form together a 5-, 6- or 7-membered heterocyclic ring containing 1 to 4 heteroatoms independently selected from N, O and S, and said ring may be substituted with one or more B; B is hydroxy, CN, R¹⁰, —COOR¹⁰, —NHCOR¹⁰, —NR¹⁰R¹¹, —CONR¹⁰R¹¹, or —OR¹⁰; R⁵ is phenyl or heteroaryl each of which is optionally substituted with one or more of —OR¹⁰, —R¹⁰, —CONR¹⁰R¹¹, —NR¹⁰R¹¹, or halogeno; R⁶ is hydrogen, or C₁₋₆alkyl; R⁷ is C₁₋₆alkyl; A is hydrogen, R⁸, or —NR⁸R⁹; R⁸ and R⁹ each independently are hydrogen, C₁₋₆alkyl; R¹⁰ and R¹¹ each independently are hydrogen, C₁₋₆alkyl, C₁₋₆alkanol, or; R¹⁰ and R¹¹ each independently are hydrogen, C₁₋₆alkyl, C₁₋₆fluoroalkyl or hydroxyC₁₋₆alkyl, or R¹⁰ and R¹¹ form together a 5-, 6- or 7-membered heterocyclic ring containing 1 to 4 heteroatoms independently selected from N, O and S, and said ring may be substituted with one or more B.

4. A compound according to any one of claims 1 to 3, wherein R¹ is phenyl optionally substituted with one or more of the following —OR³, —COOR³, —CONR³R⁴, —NHCOR³, —NR³R⁴, or —SO₂R³.

5. A compound according to claim 4, wherein R³ and/or R⁴ are each independently hydrogen, C₁₋₆alkyl, or heterocycleC₀₋₆alkyl, and said C₁₋₆alkyl may be substituted with one or more B; or R³ and R⁴ form together a 5-, 6- or 7-membered heterocyclic ring containing 1 to 4 heteroatoms independently selected from N, O and S, and said ring may be substituted with one or more B; B is CN, C₁₋₆alkyl, R¹⁰, —COOR¹⁰, —NHCOR¹⁰, —NR¹⁰R¹¹, —CONR¹⁰R¹¹, or —OR¹⁰.

6. A compound according to any one of claims 1 to 5, wherein R¹⁰ and R¹¹ each independently are hydrogen, C₁₋₆alkyl, C₁₋₆fluoroalkyl or hydroxyC₁₋₆alkyl, or R¹⁰ and R¹¹ form together a 5-, 6- or 7-membered heterocyclic ring containing 1 to 4 heteroatoms independently selected from N, O and S, and said ring may be substituted with one or more B.

7. A compound according to any one of claims 1 to 3, wherein R¹ is heteroaryl.

8. A compound according to any one of claims 1 to 7, wherein R² is NR⁵R⁶ and said R⁵ is phenyl optionally substituted with one or more R¹⁰, OR¹⁰, halogeno, and said R⁶ is hydrogen.

9. A compound according to claim 8, wherein said halogeno is chloro.

10. A compound according to any one of claims 1 to 7, wherein R² is NO₂, or NH₂.

11. A compound according to any one of claims 1 to 10, wherein A is hydrogen, R⁸, or NR⁸R⁹, and R⁸ and R⁹ each independently are hydrogen, or C₁₋₆alkyl, and said C₁₋₆alkyl may be substituted with one or more B; or R⁸ and R⁹ form together a 5-, 6- or 7-membered heterocyclic ring containing 1 to 4 heteroatoms independently selected from N, O and S, and said ring may be substituted with one or more B.

12. A compound which is:
(2-Chloro-phenyl)-(3-phenyl-1H-indazol-6-yl)-amine Hydrochloride;
Phenyl-(3-phenyl-1H-indazol-6-yl)-amine Hydrochloride;
(4-Fluoro-phenyl)-(3-phenyl-1H-indazol-6-yl)-amine Hydrochloride;
(3-Phenyl-1H-indazol-6-yl)-(4-trifluoromethyl-phenyl)-amine Hydrochloride;
(3-Phenyl-1H-indazol-6-yl)-(3-trifluoromethyl-phenyl)-amine Hydrochloride;
(3-Phenyl-1H-indazol-6-yl)-pyridin-2-yl-amine Hydrochloride;
Phenyl-[3-(1H-pyrrol-2-yl)-1H-indazol-6-yl]-amine Hydrochloride;
(2-Methoxy-phenyl)-(3-phenyl-1H-indazol-6-yl)-amine;
(3-Phenyl-1H-indazol-6-yl)-pyridin-3-yl-amine Hydrochloride;
Benzyl-(3-phenyl-1H-indazol-6-yl)-amine Hydrochloride;
Cyclopropylmethyl-(3-phenyl-1H-indazol-6-yl)-amine Hydrochloride;
Methyl-(3-phenyl-1H-indazol-6-yl)-amine Hydrochloride;
6-Nitro-3-(1H-pyrrol-2-yl)-1H-indazole hydrochloride;
6-Nitro-3-pyridin-3-yl-1H-indazole hydrochloride;
3-Furan-2-yl-6-nitro-1H-indazole hydrochloride;
Dimethyl-[4-(6-nitro-1H-indazol-3-yl)-phenyl]-amine Hydrochloride;
N-[3-(6-nitro-1H-indazol-3-yl)-phenyl]-acetamide;
3-Pyridin-3-yl-1H-indazol-6-ylamine;
3-(1H-Pyrrol-2-yl)-1H-indazol-6-ylamine hydrochloride;
3-(3-Methoxy-phenyl)-1H-indazol-6-ylamine hydrochloride;
N-(2-chlorophenyl)-3-[4-(methylsulfonyl)phenyl]-1H-indazol-6-amine Hydrochloride;
Methyl 4-{6-[(2-chlorophenyl)amino]-1H-indazol-3-yl}benzoate dihydrochloride;
4-{6-[(2-Chlorophenyl)amino]-1H-indazol-3-yl}benzoic acid dihydrochloride;
Methyl 3-{6-[(2-chlorophenyl)amino]-1H-indazol-3-yl}benzoate dihydrochloride;
3-{6-[(2-Chlorophenyl)amino]-1H-indazol-3-yl}benzoic acid dihydrochloride;
N-(2-chlorophenyl)-3-{3-[(4-methylpiperazin-1-yl)carbonyl]phenyl}-1H-indazol-6-amine;
4-{6-[(2-Chlorophenyl)amino]-1H-indazol-3-yl}-N-[3-(4-methylpiperazin-1-yl)propyl]benzamide;
4-{6-[(2-Chlorophenyl)amino]-1H-indazol-3-yl}-N-(2-morpholin-4-ylethyl)benzamide;
4-{6-[(2-Cchlorophenyl)amino]-1H-indazol-3-yl}-N-[2-(dimethylamino)ethyl]benzamide;
4-{6-[(2-Chlorophenyl)amino]-1H-indazol-3-yl}-N-[3-(dimethylamino)propyl]benzamide;
4-{6-[(2-Chlorophenyl)amino]-1H-indazol-3-yl}-N-[3-carbamoylmethyl-benzamide;
N-(2-chlorophenyl)-3-[4-(thiomorpholin-4-ylcarbonyl)phenyl]-1H-indazol-6-amine;
Methyl N-(4-{6-[(2-chlorophenyl)amino]-1H-indazol-3-yl} benzoyl)-N-methylglycinate;
1-(4-{6-[(2-Chlorophenyl)amino]-1H-indazol-3-yl} benzoyl)pyrrolidin-3-ol;
4-{6-[(2-Chlorophenyl)amino]-1H-indazol-3-yl}-N,N-bis(cyanomethyl)benzamide;
N-(2-Chlorophenyl)-3-(4-{[3-(dimethylamino)pyrrolidin-1-yl]carbonyl}phenyl)-1H-indazol-6-amine;
4-{6-[(2-Chlorophenyl)amino]-1H-indazol-3-yl}-N-[2-(dimethylamino)ethyl]-N-ethylbenzamide;
1-(4-{6-[(2-Chlorophenyl)amino]-1H-indazol-3-yl}benzoyl)piperidine-4-carboxamide;
4-{6-[(2-Chlorophenyl)amino]-1H-indazol-3-yl}-N-(2-hydroxyethyl)-N-methylbenzamide;
1-(4-{6-[(2-Chlorophenyl)amino]-1H-indazol-3-yl}benzoyl)piperidin-4-ol;
N-(2-chlorophenyl)-3-[4-(morpholin-4-ylcarbonyl)phenyl]-1H-indazol-6-amine;
3-{6-[(2-Chlorophenyl)amino]-1H-indazol-3-yl}-N-{3-[(2-hydroxyethyl)(methyl)amino]propyl}benzamide;
3-{6-[(2-Chlorophenyl)amino]-1H-indazol-3-yl}-N-(3-morpholin-4-ylpropyl)benzamide;
3-{6-[(2-Chlorophenyl)amino]-1H-indazol-3-yl}-N-[2-(diethylamino)-1-methylethyl]benzamide;
3-{6-[(2-Chlorophenyl)amino]-1H-indazol-3-yl}-N-[2-hydroxy-1-(hydroxymethyl)-1-methylethyl]benzamide;
3-{6-[(2-Chlorophenyl)amino]-1H-indazol-3-yl}-N-(2-morpholin-4-ylethyl)benzamide;
Ethyl 4-[(3-{6-[(2-chlorophenyl)amino]-1H-indazol-3-yl} benzoyl)amino]piperidine-1-carboxylate;
3-{6-[(2-Chlorophenyl)amino]-1H-indazol-3-yl}-N-(2-piperidin-1-ylethyl)benzamide;
3-{6-[(2-Chlorophenyl)amino]-1H-indazol-3-yl}-N-[2-(dimethylamino)ethyl]benzamide;
3-{6-[(2-Chlorophenyl)amino]-1H-indazol-3-yl}-N-[3-(dimethylamino)propyl]benzamide;
3-{6-[(2-Chlorophenyl)amino]-1H-indazol-3-yl}-N-(2-ethoxyethyl)benzamide;
3-{6-[(2-Chlorophenyl)amino]-1H-indazol-3-yl}-N-(2-hydroxyethyl)benzamide;
N-[2-(acetylamino)ethyl]-3-{6-[(2-chlorophenyl)amino]-1H-indazol-3-yl} benzamide;
3-{6-[(2-Chlorophenyl)amino]-1H-indazol-3-yl}-N-carbamoylmethyl-benzamide;
3-{6-[(2-Chlorophenyl)amino]-1H-indazol-3-yl}-N-(1-ethylpiperidin-3-yl)benzamide;
3-{6-[(2-Chlorophenyl)amino]-1H-indazol-3-yl}-N-(3-pyrrolidin-1-ylpropyl)benzamide;
3-{6-[(2-Chlorophenyl)amino]-1H-indazol-3-yl}-N-[3-(4-methylpiperazin-1-yl)propyl]benzamide;
3-{6-[(2-Chlorophenyl)amino]-1H-indazol-3-yl}-N-[(1-ethylpyrrolidin-2-yl)methyl]benzamide;
3-{6-[(2-Chlorophenyl)amino]-1H-indazol-3-yl}-N-(tetrahydrofuran-2-ylmethyl)benzamide;
(2-Chloro-phenyl)-(5-methyl-3-phenyl-1H-indazol-6-yl)-amine Hydrochloride;
N-(2-morpholin-4-ylethyl)-6-nitro-3-phenyl-1H-indazol-5-amine Hydrochloride;
(2-Fluoro-phenyl)-(3-phenyl-1H-indazol-6-yl)-amine Hydrochloride;
3-(4-Methanesulfonyl-phenyl)-6-nitro-1H-indazole hydrochloride;
3-Furan-3-yl-6-nitro-1H-indazole hydrochloride;
as a free base or a salt thereof.

13. A pharmaceutical formulation comprising as active ingredient a therapeutically effective amount of the compound of any one of claims 1 to 12 in association with pharmaceutically acceptable carriers or diluents.

14. A pharmaceutical formulation comprising as active ingredient a therapeutically effective amount of the compound of any one of claims 1 to 12 for use in the prevention and/or treatment of conditions associated with JNK activation.

15. A compound according to any one of claims 1 to 12 for use in therapy.

16. Use of a compound according to any one of claims 1 to 12 in the manufacture of a medicament for the prevention and/or treatment of conditions associated with JNK activation.

17. Use of a compound according to any one of claims 1 to 12 in the manufacture of a medicament for the prevention and/or treatment of conditions selected from: central or peripheral neurological degenerative disorders including Alzheimer's disease, cognitive disorders, Parkinson's disease, Huntington's disease, amyotrophic lateral sclerosis, Frontotemporal dementia Parkinson's Type, Parkinson dementia complex of Gaum, HIV dementia, corticobasal degeneration, dementia pugilistica, Down's syndrome, postencephelatic parkinsonism, progressive supranuclear palsy, Pick's Disease, Niemann-Pick's Disease, epilepsy, a peripheral neuropathy, spinal cord injury, head trauma; and
cancer.

18. The use according to claim 17 wherein said condition is Alzheimer's disease.

19. Use of a compound according to any one of claims 1 to 12 in the manufacture of a medicament for the prevention and/or treatment of conditions associated with inhibiting the expression of inducible pro-inflammatory proteins.

20. Use of a compound according to any one of claims 1 to 12 in the manufacture of a medicament for the prevention and/or treatment of conditions selected from edema, analgesia, fever and pain, such as neuromuscular pain, headache, cancer pain, dental pain and arthritis pain.

21. A method of treating or preventing conditions associated with JNK activation comprising the administration of a therapeutically effective amount of a compound of Formula I according to any one of claims 1 to 12 to a mammal in need thereof.

22. A method of treating or preventing conditions selected from central or peripheral neurological degenerative disorders including Alzheimer's disease, cognitive disorders, Parkinson's disease, Huntington's disease, amyotrophic lateral sclerosis, Frontotemporal dementia Parkinson's Type, Parkinson dementia complex of Gaum, HIV dementia, corticobasal degeneration, dementia pugilistica, Down's syndrome, postencephelatic parkinsonism, progressive supranuclear palsy, Pick's Disease, Niemann-Pick's Disease, epilepsy, a peripheral neuropathy, spinal cord injury, head trauma; and cancer comprising the administration of a therapeutically effective amount of a compound of Formula I according to any one of claims 1 to 12 to a mammal in need thereof.

23. The method according to claim 22, wherein said condition is Alzheimer's disease.

24. A method of treating or preventing associated with inhibiting the expression of inducible pro-inflammatory proteins comprising the administration of a therapeutically effective amount of a compound of Formula I according to any one of claims 1 to 12 to a mammal in need thereof.

25. The method according to claim 24, wherein the condition is selected from edema, analgesia, fever and pain, such as neuromuscular pain, headache, cancer pain, dental pain and arthritis pain.

26. A compound according to formula II: 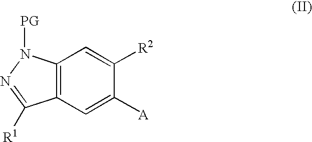
wherein:
R¹, R² and A are as defined in claim 1; and
PG is an amino protecting group;
as a free base, salt, solvate or solvate of salt therof.

27. A process for the preparation of a compound of Formula I comprising the de-protection of a compound of Formula II 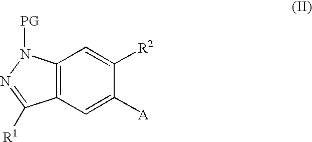
in which R¹, R² and A are as defined in claim 26 and PG is an amino protecting group.

28. The use compound according to Formula II in claim 26 for the preparation of a compound of formula I as defined in any one of claims 1 to 12.
